(19) United States
(12) Patent Application Publication
(10) Pub. No.: US 2021/0381059 A1
(43) Pub. Date: Dec. 9, 2021

(54) USE OF LONG NON-CODING RNA FOR THE DIAGNOSIS OF PROSTATE CANCER

(71) Applicants: INSTITUT CURIE, PARIS CEDEX 05 (FR); CENTRE NATIONAL DE LA RECHERCHE SCIENTIFIQUE, PARIS (FR); UNIVERSITE PARIS-SACLAY, GIF-SUR-YVETTE (FR); SORBONNE UNIVERSITE, PARIS (FR)

(72) Inventors: ANTONIN MORILLON, PARIS (FR); DANIEL GAUTHERET, GIF SUR YVETTE (FR); MARINA PINSKAYA, ORSAY (FR)

(21) Appl. No.: 17/287,132

(22) PCT Filed: Oct. 24, 2019
(86) PCT No.: PCT/EP2019/078974

§ 371 Date: Apr. 21, 2021

(30) Foreign Application Priority Data
Oct. 25, 2018 (EP) 18306391.6

(51) Int. Cl. C12Q 1/6886

(52) U.S. Cl. CPC C12Q 1/6886; C12Q 2600/118; C12Q 2600/158

(57) ABSTRACT

The present invention provides long noncoding RNAs (lncRNAs) that allow to diagnose prostate cancer much more accurately than the existent non-invasive diagnostic tools. So, this invention relates to the use of at least one of these lncRNA or a combination thereof as a diagnosis marker for prostate cancer. It also relates to an in vitro method for prostate cancer diagnosis of a subject as well as to a kit for performing this method.

### FIELD OF THE INVENTION

The present invention relates to the field of medicine, in particular of oncology. It provides new diagnostic markers in prostate cancer.

### BACKGROUND OF THE INVENTION

Over the decade, cancer of the prostate has become the most commonly diagnosed malignancy among men and the second leading cause of male cancer deaths in the western population, following lung cancer.

Early detection and treatment of prostate cancer before it has spread from the prostate gland reduces the mortality of the disease. This realization has prompted increasing efforts for early diagnosis and treatment. Indeed, the American Cancer Society recommend that male population at large undergo annual screening for prostate cancer beginning at age 50. The recommended age for screening is lowered to 40 for men giving a family history of prostate cancer or other risk factors.

Early screening and detection could also translate into a reduction in the healthcare burden, as early treatment can be less radical, more successful and therefore provided at a lower cost per treated patient. The key to accomplish this goal is to provide better differential diagnostic tools.

Nowadays, screening for prostate cancer involves mainly palpation of the prostate by digital rectal examination and assay of plasma levels of prostate specific antigen (PSA). PSA is a serine protease produced by the prostatic epithelium that is normally secreted in the seminal fluid to liquefy it. Disruption of the anatomy integrity of the prostate gland can compromise the cellular barriers that normally restrict PSA to within the duct system of the prostate, allowing it to disperse into blood or urine. A number of conditions can result in this leakage of PSA, including inflammation of the prostate, urinary retention, prostatic infection, benign prostatic hyperplasia, and prostate cancer. It is therefore not surprising that screening of serum PSA as an indicator of prostate cancer is not absolutely predictive.

This low level of specificity results in additional more invasive and costly diagnostic procedures. Indeed, the normal procedure for a subject positive to PSA and palpation is to proceed to a biopsy which consists in 10 to 15 extractions of prostatic tissue. Among the subjects tested, only 45% appear to really have a cancer and 10% develop a prostate infection after the biopsy. Biopsies, when unnecessary, are also very traumatic for the patients. The psychological impact of being diagnosed as positive until proven as a false positive should not be understated either. Moreover, even a biopsy is not always 100% certain, and a second biopsy procedure is often required.

High-throughput RNA sequencing has produced catalogues of long non-coding RNAs (lncRNAs) with now 58648 lncRNAs annotated (Iyer M K et al, Nat Genet, 2015, 47, pp. 199-208). LncRNA are RNA of at least 200 nucleotides long, cell type/tissue specific and poorly conserved during evolution. Among different classes of lncRNAs, natural antisense transcripts are the less described with only 4200 annotated antisense lncRNAs (Derrien T et al, Genome Res, 2012, 22, pp. 1775-1789), certainly due to their low expression. In addition, most genome-wide studies were focused on lncRNAs without addressing their strand-specificity explaining a lack of systematic characterization of antisense transcriptome.

LncRNAs have been shown to exhibit oncogenic or tumor suppressive functions in cancer biology. Being highly tissue specific and deregulated during tumorigenesis, an increasing number of lncRNAs were also proposed as biomarkers for diagnostic, prognostic and/or monitoring purposes. So far, only one lncRNA, PCA3, received the FDA approval for decisions to perform a biopsy in a prostate cancer (Groskopf J et al, Clinical chemistry, 2006, 52, pp. 1089-1095). PCA3 is proposed as an optional urinary test to provide more accurate metrics regarding repeated biopsies. Although addition of the PCA3 score globally improves diagnostic accuracy, it remains inaccurate in some tumours, especially among old patients and patients with advanced cancer and in discrimination between low risk and aggressive disease, since its expression becomes highly heterogeneous and may go down in severe PCa cases. PCa transcriptome has been vastly explored by The Cancer Genome Atlas (TCGA) consortium and others to identify numerous PCa associated lncRNAs (PCAT family) such as PCAT1, PCAT7 or SChLAP1/PCAT18. Regardless the richness of clinical specimens, the experimental setup based on poly(A) selection of RNA species, unstranded cDNA sequencing protocols and poor sequencing depth of the TCGA collection limits largely the sensitivity and accuracy in discovery of lncRNA species. The disclosure WO 2017/103047 relates to three lncRNA useful for prostate cancer diagnosis.

In view of the fact that advanced prostate cancer remains a life threatening disease reaching a very significant proportion of the male population and the number of unnecessary biopsies, there is a strong need to provide the most specific, selective, and rapid prostate cancer detection methods and kits. Thus, further investigations are still needed to identify new biomarkers and to evaluate their clinical performances. The present invention seeks to meet these and other needs.

### SUMMARY OF THE INVENTION

The inventors have discovered new lncRNAs biomarkers of prostate cancer, particularly signatures comprising combinations of lncRNAs as biomarkers that present more robustness and better diagnostic precision of prostate cancer than the actual diagnostic PCA3 marker.

The invention concerns the use of at least one biomarker for prostate cancer diagnosis, wherein said at least one biomarker is a lncRNA (long non coding RNA) or lncRNA fragment thereof of at least 30 nucleotides, the lncRNA or lncRNA fragment thereof of at least 30 nucleotides being selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20, SEQ ID NO: 21, SEQ ID NO: 22 and any fragment thereof of at least 30 nucleotides.

In another aspect the invention concerns an in vitro method for prostate cancer diagnosis of a subject, wherein the method comprises the step of determining the amount of at least one biomarker in a biological sample from said subject, said at least one biomarker being selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20, SEQ ID NO: 21, SEQ ID NO: 22 and any fragment thereof of at least 30 nucleotides, and wherein an increased amount of the at least one biomarker is indicative of prostate cancer.

Preferably, the at least one biomarker is selected the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 20, SEQ ID NO: 21 and any fragment thereof of at least 30 nucleotides.

Even more preferably, the at least one biomarker is used in combination with at least one additional biomarker selecting from the group consisting of SEQ ID NO: 15, SEQ ID NO: 16, SEQ ID NO: 17, SEQ ID NO: 18, SEQ ID NO: 19, SEQ ID NO: 23, SEQ ID NO: 24 and any fragment thereof of at least 30 nucleotides.

Particularly, at least three, four, five, six, seven or eight biomarkers are used in combination and wherein said combination are selected in any one of the following groups consisting of

SEQ ID NO: 1, SEQ ID NO: 2, SEQ ID NO: 3, SEQ ID NO: 4, SEQ ID NO: 10, SEQ ID NO: 11 and a fragment of at least 30 nucleotides thereof;

SEQ ID NO: 1, SEQ ID NO: 2, SEQ ID NO: 3, SEQ ID NO: 4, SEQ ID NO: 5, SEQ ID NO: 10, SEQ ID NO: 14 and a fragment of at least 30 nucleotides thereof;

SEQ ID NO: 1, SEQ ID NO: 2, SEQ ID NO: 3, SEQ ID NO: 10, SEQ ID NO: 11, SEQ ID NO: 14, and a fragment of at least 30 nucleotides thereof; and

SEQ ID NO: 1, SEQ ID NO: 2, SEQ ID NO: 3, SEQ ID NO: 4, SEQ ID NO: 5, SEQ ID NO: 10, SEQ ID NO: 11, SEQ ID NO: 14 and a fragment of at least 30 nucleotides thereof.

Particularly, the combination comprises or consists of any one of the following combinations of biomarkers:

SEQ ID NO: 1, SEQ ID NO: 2, SEQ ID NO: 3, SEQ ID NO: 4, SEQ ID NO: 10, SEQ ID NO: 11 and a fragment of at least 30 nucleotides thereof;

SEQ ID NO: 1, SEQ ID NO: 2, SEQ ID NO: 3, SEQ ID NO: 4, SEQ ID NO: 5, SEQ ID NO: 10, SEQ ID NO: 14 and a fragment of at least 30 nucleotides thereof;

SEQ ID NO: 1, SEQ ID NO: 2, SEQ ID NO: 3, SEQ ID NO: 10, SEQ ID NO: 14, and a fragment of at least 30 nucleotides thereof;

SEQ ID NO: 1, SEQ ID NO: 2, SEQ ID NO: 3, SEQ ID NO: 10 and a fragment of at least 30 nucleotides thereof; and

SEQ ID NO: 1, SEQ ID NO: 2, SEQ ID NO: 3, SEQ ID NO: 10, SEQ ID NO: 11, SEQ ID NO: 14 and a fragment of at least 30 nucleotides thereof.

Particularly, the above mentioned combination further comprises one, two, three or four biomarkers selected in the group consisting of SEQ ID NO: 15, SEQ ID NO: 16, SEQ ID NO:17, SEQ ID NO: 19 and any fragment of at least 30 nucleotides thereof.

Preferably, the combination comprises consists of the following combinations of biomarkers:

SEQ ID NO: 1, SEQ ID NO: 2, SEQ ID NO: 3, SEQ ID NO: 4, SEQ ID NO: 10, SEQ ID NO: 11, SEQ ID NO: 15, SEQ ID NO: 16, SEQ ID NO: 19 and a fragment of at least 30 nucleotides thereof, and optionally SEQ ID NO: 22 or a fragment of at least 30 nucleotides thereof;

SEQ ID NO: 1, SEQ ID NO: 2, SEQ ID NO: 3, SEQ ID NO: 4, SEQ ID NO: 5, SEQ ID NO: 10, SEQ ID NO: 14, SEQ ID NO: 15, SEQ ID NO: 16, SEQ ID NO: 17, SEQ ID NO: 19 and a fragment of at least 30 nucleotides thereof;

SEQ ID NO: 1, SEQ ID NO: 2, SEQ ID NO: 3, SEQ ID NO: 10, SEQ ID NO: 11, SEQ ID NO: 14, SEQ ID NO: 15, SEQ ID NO: 16, SEQ ID NO: 17, SEQ ID NO: 19 and a fragment of at least 30 nucleotides thereof, and optionally SEQ ID NO: 22 and/or SEQ ID NO: 23 and/or SEQ ID NO: 24 or a fragment of at least 30 nucleotides thereof;

SEQ ID NO: 1, SEQ ID NO: 2, SEQ ID NO: 3, SEQ ID NO: 10, SEQ ID NO: 11, SEQ ID NO: 14, SEQ ID NO: 15, SEQ ID NO: 16, SEQ ID NO: 17, SEQ ID NO: 19 and a fragment of at least 30 nucleotides thereof; and

SEQ ID NO: 1, SEQ ID NO: 2, SEQ ID NO: 3, SEQ ID NO: 10, SEQ ID NO: 14, SEQ ID NO: 15, SEQ ID NO: 16, SEQ ID NO: 19 and a fragment of at least 30 nucleotides thereof; and

SEQ ID NO: 1, SEQ ID NO: 2, SEQ ID NO: 3, SEQ ID NO: 10, SEQ ID NO: 15, and SEQ ID NO: 16 and a fragment of at least 30 nucleotides thereof.

Particularly, the use or method disclosed herein further discriminate high- and intermediate-risk of developing a prostate cancer versus low-risk of developing a prostate cancer in a subject. Preferably, the subject is a mammal, preferably a human, more preferably a man of at least 40 years old.

The sample used is a body fluid, preferably a urine or blood sample, more preferably a urine sample. Preferably, the biomarker amount is determined by amplification or by hybridization, preferably by quantitative RT-PCR or by the Nanostring method, preferably by the Nanostring method.

The invention also relates to a kit for the diagnosis of prostate cancer in a subject, wherein the kit comprises (i) probes and/or primers capable to specifically hybridize to at least one biomarker selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ TD NO: 12, SEQ ID NO: 13, SEQ ID NO: 14, SEQ ID NO:20 and SEQ ID NO: 21; and optionally, a leaflet providing guidelines to use such a kit.

Preferably, the kit further comprises probes and/or primers for the detection of additional prostate cancer biomarkers, preferably probes and/or primers for the detection of antisense lncRNAs prostate cancer biomarkers, more preferably probes and/or primers capable to specifically hybridize to at least one biomarker selected from the group consisting of SEQ ID NO: 15, SEQ ID NO: 16, SEQ ID NO: 17, SEQ ID NO: 18, SEQ ID NO: 19, SEQ ID NO: 22, SEQ ID NO: 23 and SEQ ID NO: 24.

The invention also concerns the use of such kit in the diagnosis of prostate cancer in a subject, preferably a human, even more preferably an adult man of at least 40 years old.

Finally, the invention relates to an isolated nucleic acid comprising or consisting of a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO:20 and SEQ ID NO: 21 and any fragment thereof of at least 30 nucleotides or a complementary sequence thereof, wherein the nucleic acid sequence has a length shorter than 3500 nucleotides.

### BRIEF DESCRIPTION OF THE DRAWINGS

FIG. 1: Expression of contigs in TCGA-PRAD and PAIR specimens. A. experimental workflow for discovery and validation of contigs expression by different technologies in two independent cohorts. B. Box-plot of Log 10 (counts) of PCA3 and 23 contigs in 144 PAIR samples by NanoString. C. in 24 PAIR specimens by total stranded RNA-seq. D. and 557 TCGA-PRAD specimens by polyA+unstranded RNA-seq. Normal tissue—in blue, tumor—in red.

FIG. 2. Box-plot of Log 10(expression) of housekeeping protein-coding genes in 9 normal and 135 tumor specimens of the PAIR cohort by NanoString nCounter assay.

FIG. 3. Risk classification of prostate tumors according to their clinic-pathological features. A, Risk prognosis classification criteria according to D'Amico and PSA independent protocol applied in-house. B, Pie charts of different risk groups of the TCGA-PRAD specimens according to in-house and D'Amico protocols. C, Risk classification and recurrence status of prostate specimens from TCGA-PRAD and PAIR cohorts used in this study.

FIG. 4. Box-plot of Log 10(expression) of PCA3 and DE-kupl contigs in prostate specimens of PAIR (A) and TCGA (B) cohorts depending on tumor risk and recurrence status assessed by NanoString or by polyA+unstranded RNA-seq, respectively. PCA3 is marked in orange, and the contigs showing insignificant expression change between normal and tumor specimens are in blue. Probes are ordered by decreasing FC of mean expression.

FIG. 5. ROC analysis of PCA3 and DE-kupl contigs diagnostic ability in PAIR NanoString (A), PAIR RNA-seq (B) and TCGA-PRAD (C) datasets: histogram plots of AUC scores in comparison between normal and tumor tissues ranked according to p-values.

FIG. 6. Diagnostic performance of PCA3 and multiplex signatures inferred from logistic lasso regression of complete (Signature P1 and T1) and prostate specific (Signature P2 and T2) set of probes. A, ROC analysis of PCA3 along and the association of probes across PAIR and TCGA-PRAD cohorts. B, Venn diagram representing the intersection of TCGA-PRAD and PAIR signatures. Signatures:

P1 (n=10): ctg_73782, ctg_104447, PCA3, ctg_105149, ctg_117356, ctg_17297, ctg_28650, ctg_44030, ctg_512, ctg_61472.

P2 (n=10): ctg_73782, ctg_104447, PCA3, ctg_105149, ctg_111348, ctg_17297, ctg_2815, ctg_44030, ctg_512, ctg_61472.

T1 (n=13): PCA3, ctg_105149, ctg_111158, ctg_25348, ctg_104447, ctg_23999, ctg_61472, ctg_28650, ctg_117356, ctg_44030, ctg_512, ctg_17297, ctg_111348.

T2 (n=10): PCA3, ctg_105149, ctg_104447, ctg_23999, ctg_61472, ctg_117356, ctg_44030, ctg_512, ctg_17297, ctg_111348.

PTC (n=6): ctg_105149, ctg_104447, ctg_17297, ctg_44030, ctg_512, ctg_61472.

### DETAILED DESCRIPTION OF THE INVENTION

### Introduction

Recent data have uncovered a great number of long non-coding RNAs (lncRNAs). However, until now, lncRNAs have been poorly studied despite their importance in disease and tissue specificity. By performing hypothesis free RNA-sequencing and computational analysis, the inventors have identified 17 lncRNAs that are highly specific of prostate cancers and can be used as diagnostic tools for cancerous lesions. These lncRNAs used as prostate cancer biomarkers appear to present more robustness and better diagnostic precision than the actual diagnostic PCA3 marker, recently approved by FDA.

The actual diagnosis procedure, i.e. prostate palpation and PSA assay followed by biopsy, leads to 55% of misadapted biopsies (the subjects tested doesn't have a cancer).

Regression analysis identified a combination of probes targeting lncRNA allowing at least 92% true positive detection of PCa in different datasets.

This new diagnosis test is highly specific (more than 90% of the biopsies are true positives), rapid (results are obtained the day of the urine collection), and cost effective (unnecessary biopsies are avoided).

### Definitions

As used herein, the term “diagnosis markers” and “biomarkers” are interchangeable and refer to biological parameters that aid the diagnosis of a disease and/or permit the identification of patients suffering from a disease. It is a measurable indicator of the presence of this disease. This term refers particularly to “tumor diagnosis markers”. Tumor diagnosis markers are substances that are produced by cancer or by other cells of the body in response to cancer. Most tumor diagnosis markers are produced by normal cells in the absence of cancer as well; however, they are produced at much higher levels in cancerous conditions. These substances can be found in the blood, urine, stool, tumor tissue, or other tissues or bodily fluids of some patients with cancer.

As used herein, the term “lncRNAs” refers to long non-coding RNAs that generally have a length above 200 nucleotides, typically between 200 and 20 kb. Preferred population of lncRNAs within the context of the present invention is represented by long intergenic non coding RNAs (lincRNA) and antisense lncRNA (aslncRNA). The term “lincRNA” refers to long intergenic non-coding RNA. LlncRNA has an exon-intron-exon structure, similar to protein-coding genes, but does not encompass open-reading frames and does not code for proteins.

The term “antisense lncRNA”, “(as)lncRNA”, and “Natural Antisense Transcripts (NATs)” refers to a subset of long non-coding RNAs being antisense transcripts. An antisense lncRNA is a lncRNA produced from the non-coding strand of a given gene, which means that the sequence of an antisense lncRNA is complementary to the pre-mRNA sequence of the said given gene. Therefore, the sequence of the antisense lncRNA cannot be complementary to artificial sequences, such as morpholinos, that are antisense to the said given gene. Quantitatively, lncRNAs demonstrate about 10-fold lower abundance than mRNAs in a population of cells, which is explained by higher cell-to-cell variation of expression levels of lncRNA genes in the individual cells, when compared to protein-coding genes. In general, the majority (˜78%) of lncRNAs are characterized as tissue-specific, as opposed by only ˜19% of mRNAs. In addition to higher tissue specificity, lncRNAs are characterized by higher developmental stage specificity, and cell subtype specificity in heterogeneous tissues.

Long non-coding RNAs are non-protein coding, actively transcribed single-stranded RNAs. Mainly nuclear, lncRNA transcripts are subjected to processing in a manner analogous to mRNAs: they are capped, polyadenylated and frequently spliced. They are also cell type/tissue specific and poorly conserved during evolution. Their length, more than 200 nucleotides, distinguishes lncRNAs from small regulatory RNAs such as microRNAs (miRNAs), short interfering RNAs (siRNAs), Piwi-interacting RNAs (piRNAs), small nucleolar RNAs (snoRNAs), and other short RNAs. The term “pre-mRNA”, as used herein, refers to the nascent mRNA, prior to its maturation (capping, polyadenylation and splicing).

As used herein, the term “splicing” refers to a modification of a pre-RNA transcript in which introns are removed and exons are joined. “Alternative splicing” refers to a particular splicing process that can create a range of unique RNA splicing products from the same pre-RNA. Alternative splicing can occur in many ways, exons can be extended or skipped, or introns can be retained.

As used herein, the terms “lncRNA biomarker” or “lncRNA diagnosis biomarker” refer to lncRNA or fragment thereof that aid the diagnosis of a disease and/or permit the identification of patients suffering from a disease, such as prostate cancer. For example, the presence or the amount of a lncRNA biomarker in a biological sample can be indicative of the presence of a disease. “LncRNA biomarker” can particularly be targeted by probes to detect their presence or measure their amount in a biological sample.

As used herein, the terms “lncRNA fragment” refer to a fragment or piece of the lncRNA sequence. Preferably, a “lncRNA fragment” is a sequence of consecutive nucleotides, generally with a sequence size shorter or smaller than the corresponding lncRNA. For example, when a lncRNA fragment is of at least 30 nucleotides, it refers to 30 consecutive nucleotides of the sequence from the corresponding lncRNA.

The term “cancer” or “tumor”, as used herein, refers to the presence of cells possessing characteristics typical of cancer-causing cells, such as uncontrolled proliferation, and/or immortality, and/or metastatic potential, and/or rapid growth and/or proliferation rate, and/or certain characteristic morphological features. This term includes early stage, localized cancer, later stage, locally advanced cancer; and metastatic stage cancer in any type of subject. In particular, the term encompasses prostate cancer at any stage of progression.

As used herein, the term “diagnosis” refers to the determination as to whether a subject is likely to be affected with a disease such as prostate cancer. The skilled artisan often makes a diagnosis on the basis of one or more diagnosis markers or biomarkers, the presence, absence, expression level or amount of which is indicative of the presence or absence of the prostate cancer. By “diagnosis” is also intended to refer to the providing of information useful for diagnosis.

As used herein, the term “treatment”, “treat” or “treating” refers to any act intended to ameliorate the health status of patients such as therapy, prevention, prophylaxis and retardation of the disease.

As used herein, the term “effective amount” or “therapeutic effective amount” refers to a quantity of a pharmaceutical composition which prevents, removes or reduces the deleterious effects of the prostate cancer.

As used herein, the term “nucleic acid molecule” or “nucleic acid” refer to an oligonucleotide, nucleotide or polynucleotide. A nucleic acid molecule may include deoxyribonucleotides, ribonucleotides, modified nucleotides or nucleotide analogs in any combination.

As used herein, the term “nucleotide” refers to a chemical moiety having a sugar (modified, unmodified, or an analog thereof), a nucleotide base (modified, unmodified, or an analog thereof), and a phosphate group (modified, unmodified, or an analog thereof).

Nucleotides include deoxyribonucleotides, ribonucleotides, and modified nucleotide analogs including, for example, locked nucleic acids (“LNAs”), peptide nucleic acids (“PNAs”), L-nucleotides, ethylene-bridged nucleic acids (“ENAs”), arabinoside, and nucleotide analogs (including abasic nucleotides).

As used herein, the term “sequence identity” or “identity” refers to an exact nucleotide to nucleotide correspondence of two polynucleotides. Percent of identity can be determined by a direct comparison of the sequence information between two molecules by aligning the sequences, counting the exact number of matches between the two aligned sequences, dividing by the length of the shorter sequence, and multiplying the result by 100.

As used herein, the term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

The term “hybridization”, as used herein, refers to “nucleic acid hybridization”. Nucleic acid hybridization depends on a principle that two single-stranded nucleic acid molecules that have complementary base sequences will form a thermodynamically favored double-stranded structure if they are mixed under the proper conditions. The double-stranded structure will be formed between two complementary single-stranded nucleic acids even if one is immobilized.

As used herein, the term “amplification” refers to the amplification of a sequence of a nucleic acid. It's a method for generating large amounts of a target sequence. In general, one or more amplification primers are annealed to a nucleic acid sequence. Using appropriate enzymes, sequences found adjacent to, or in between the primers are amplified.

The term “probe”, as used herein, means a strand of DNA or RNA of variable length (about 20-1000 bases long) which can be labelled. The probe is used in DNA or RNA samples to detect the presence of nucleotide sequences (the DNA or RNA target) that are complementary to the sequence in the probe.

The term “primer”, as used herein, means a strand of short DNA sequence that serves as a starting point for DNA synthesis. The polymerase starts polymerization at the 3′-end of the primer, creating a complementary sequence to the opposite strand. “PCR primers” are chemically synthesized oligonucleotides, with a length between 10 and 30 bases long, preferably about 20 bases long.

As used herein, the term “complementary DNA” (cDNA) refers to recombinant nucleic acid molecules synthetized by reverse transcription of a RNA molecule, for example an lncRNA.

As used herein, the term “hybridizing conditions” is intended to mean those conditions of time, temperature, and pH, and the necessary amounts and concentrations of reactants and reagents, sufficient to allow at least a portion of complementary sequences to anneal with each other. As it is well known in the art, the time, temperature, and pH conditions required to accomplish hybridization depend on the size of the oligonucleotide probe or primer to be hybridized, the degree of complementarity between the oligonucleotide probe or primer and the target, the nucleotide type (e.g., RNA, or DNA) of the oligonucleotide probe or primer and the target, and the presence of other materials in the hybridization reaction mixture. The actual conditions necessary for each hybridization step are well known in the art or can be determined without undue experimentation. General parameters for specific (i.e., stringent) hybridization conditions for nucleic acids are described in Sambrook, et al., Molecular Cloning: A Laboratory Manual (3rd Edition, 2001). One of skills in the art will in particular appreciate that as the oligonucleotides become shorter, it may become necessary to adjust their length to achieve a relatively uniform melting temperature for satisfactory hybridization results.

The terms “quantity,” “amount,” and “level” are used interchangeably herein and may refer to an absolute quantification of a molecule in a sample, or to a relative quantification of a molecule in a sample, i.e., relative to another value such as relative to a reference value as taught herein, or to a range of values for the biomarker. These values or ranges can be obtained from a single patient or from a group of patients.

Long noncoding RNAs (lncRNAs) that may be used as cancer biomarkers and methods of diagnosing, prognosing and monitoring prostate cancer are provided herein.

In a first aspect, the invention concerns the use of at least one biomarker for prostate cancer diagnosis, wherein said at least one biomarker is a lncRNA selected from the group consisting of SEQ ID NOs: 1-14 and 20-22 or a fragment of at least 30 nucleotides thereof. Preferably, said at least one biomarker is used in combination with an additional biomarker selected from the group consisting of SED ID NO: 15-19 and 23-24 or a fragment thereof of at least 30 nucleotides. Particularly, the present invention also provides the use of the lncRNA biomarker disclosed herein as prostate cancer clinical staging index, useful to discriminate Low-risk (LR), Intermediate-risk (IR) and High-risk (HR) of developing a prostate cancer.

In a second aspect, the present invention also concerns an in vitro method for prostate cancer diagnosis of a subject, wherein the method comprises the step of determining the amount of at least one lncRNA biomarker selected from the group consisting of SEQ ID NOs: 1-14 and 20-22 or of a fragment of at least 30 nucleotides thereof in a biological sample from said subject, and wherein an increased amount of the at least one said lncRNA biomarker is indicative of prostate cancer. Preferably, the method further comprises the step of determining the amount of an additional biomarker selected from the group consisting of SED ID NO: 15-19 and 23-24 or a fragment thereof of at least 30 nucleotides.

### LncRNA

In a first aspect, the invention provides new lncRNAs of SEQ ID NOS: 1-9 and 20-21. Therefore, the invention relates to a nucleic acid comprising or consisting of a sequence selected from the group consisting of SEQ ID NOS: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20, SEQ ID NO: 21, and SEQ ID NO: 22, and any fragment thereof of at least 30 nucleotides or a complementary sequence thereof. Preferably, the nucleic acid has a length shorter than 3500, 3000, 2500, 2000, 1500, 1000, 900, 800, 700, 600, 500, 400, 300 or 200 nucleotides. Optionally, the fragment has at least 35, 40, 45, 50 or 60 nucleotides. Preferably, the nucleic acid or the fragment thereof consists in consecutive nucleotides. In one particular aspect, the invention relates to a nucleic acid comprising or consisting of a sequence selected from the group consisting of SEQ ID NOS: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 20, SEQ ID NO: 21, and any fragment thereof of at least 30 nucleotides or a complementary sequence thereof.

In a second aspect, the invention relates to the use of at least one biomarker for prostate cancer diagnosis, wherein said at least one biomarker is a lncRNA or lncRNA fragment of at least 30 nucleotides selected among the lncRNA identified by the inventors as useful for the prostate cancer diagnosis, in particular the lncRNAs of SEQ ID NOS: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20, SEQ ID NO: 21, and SEQ ID NO: 22. In a particular aspect, the lncRNAs are selected in the group consisting of SEQ ID NO: 1, SEQ ID NO: 2, SEQ ID NO: 3, SEQ ID NO: 4, SEQ ID NO: 5, SEQ ID NO: 6, SEQ TD NO: 7, SEQ ID NO: 8, SEQ ID NO: 9, SEQ ID NO: 20, and SEQ ID NO: 21.

As used herein, the term “lncRNA biomarker” includes variants of the nucleic acid sequences described herein (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, preferably SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 20, and SEQ ID NO: 21), particularly any variants that have at least 90%, at least 95%, at least 96%, at least 97%, at least 98%, at least 99% homology thereof.

The above-mentioned use and method refer to a lncRNA biomarker or a fragment thereof. As used herein, the term “fragment” refers to a portion of a lncRNA constituted of consecutive nucleotides. Preferably, said fragment has a length of at least 30 nucleotides, preferably of at least 35, 40, 45, or 50 nucleotides, more preferably of at least 50, 60, 70, 80, 90, 100, 150, 200, 250, 500, 750, or 1000 nucleotides. Preferably, said fragment has a length of at least 100 nucleotides. Even more preferably said fragment has a length not longer than 200 nucleotides. Preferably, the fragment is located in an exonic sequence of a lncRNA and, more preferably in a sequence combining at least two exons.

In one embodiment, the invention refers to the use or method for prostate cancer diagnosis, wherein said at least one biomarker is a lncRNA or lncRNA fragment of at least 30 nucleotides, the lncRNA being selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20, SEQ ID NO: 21 and SEQ ID NO: 22. In a particular embodiment, the lncRNA is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 20 and SEQ ID NO: 21.

In a preferred embodiment, the above mentioned use and method relate to a combination of at least 2, at least 3, at least 4 at least 5, at least 6, at least 7 at least 8, at least 9, at least 10, at least 11, at least 12, at least 13, at least 14, at least 15, at least 16, or at least 17 lncRNA biomarkers or a fragment of at least 30 nucleotides thereof selected in the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20, SEQ ID NO: 21 and SEQ ID NO: 22. In an additional embodiment, the above mentioned use and method relate to a combination of at least 2, at least 3, at least 4 at least 5, at least 6, at least 7 at least 8, at least 9, at least 10, or at least 11 lncRNA biomarkers or a fragment of at least 30 nucleotides thereof selected in the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 20 and SEQ ID NO: 21.

Preferably, the at least one biomarker as mentioned above can be used in combination with at least one additional biomarker selecting from the group consisting of SEQ ID NO: 15, SEQ ID NO: 16, SEQ ID NO: 17, SEQ ID NO: 18, SEQ ID NO: 19, SEQ ID NO: 23, SEQ ID NO: 24 and any fragment thereof of at least 30 nucleotides.

In one aspect of the use or method for prostate cancer diagnosis, the biomarkers are used in a combination of at least three, four, five, six, seven or eight biomarkers and wherein said combination are selected in any one of the following groups consisting of SEQ ID NO: 1, SEQ ID NO: 2, SEQ ID NO: 3, SEQ ID NO: 4, SEQ ID NO: 10, SEQ ID NO: 11 and a fragment of at least 30 nucleotides thereof;

SEQ ID NO: 1, SEQ ID NO: 2, SEQ ID NO: 3, SEQ ID NO: 4, SEQ ID NO: 5, SEQ ID NO: 10, SEQ ID NO: 14 and a fragment of at least 30 nucleotides thereof;

SEQ ID NO: 1, SEQ ID NO: 2, SEQ ID NO: 3, SEQ ID NO: 10, SEQ ID NO: 11, SEQ ID NO: 14, and a fragment of at least 30 nucleotides thereof;

SEQ ID NO: 1, SEQ ID NO: 2, SEQ ID NO: 3, SEQ ID NO: 10 and a fragment of at least 30 nucleotides thereof; and

SEQ ID NO: 1, SEQ ID NO: 2, SEQ ID NO: 3, SEQ ID NO: 4, SEQ ID NO: 5, SEQ ID NO: 10, SEQ ID NO: 11, SEQ ID NO: 14 and a fragment of at least 30 nucleotides thereof.

More specifically, the combination may comprise or consist of any one of the following combinations of biomarkers:

SEQ ID NO: 1, SEQ ID NO: 2, SEQ ID NO: 3, SEQ ID NO: 4, SEQ ID NO: 10, SEQ ID NO: 11 and a fragment of at least 30 nucleotides thereof;

SEQ ID NO: 1, SEQ ID NO: 2, SEQ ID NO: 3, SEQ ID NO: 4, SEQ ID NO: 5, SEQ ID NO: 10, SEQ ID NO: 14 and a fragment of at least 30 nucleotides thereof;

SEQ ID NO: 1, SEQ ID NO: 2, SEQ ID NO: 3, SEQ ID NO: 10, SEQ ID NO: 11, SEQ ID NO: 14, and a fragment of at least 30 nucleotides thereof;

SEQ ID NO: 1, SEQ ID NO: 2, SEQ ID NO: 3, SEQ ID NO: 10 and a fragment of at least 30 nucleotides thereof; and

SEQ ID NO: 1, SEQ ID NO: 2, SEQ ID NO: 3, SEQ ID NO: 4, SEQ ID NO: 5, SEQ ID NO: 10, SEQ ID NO: 11, SEQ ID NO: 14, and a fragment of at least 30 nucleotides thereof.

Optionally, the combination may further comprise one, two, three or four biomarkers selected in the group consisting of SEQ ID NO: 15, SEQ ID NO: 16, SEQ ID NO: 17, SEQ ID NO: 19 and any fragment of at least 30 nucleotides thereof.

Optionally, the combination may further comprise one, two, three or four biomarkers selected in the group consisting of SEQ ID NO: 20, SEQ ID NO: 21, SEQ ID NO: 22, SEQ ID NO: 23, SEQ ID NO: 24, and any fragment of at least 30 nucleotides thereof.

For instance, the combination may comprise or consist of any one of the following combinations of biomarkers:

SEQ ID NO: 1, SEQ ID NO: 2, SEQ ID NO: 3, SEQ ID NO: 4, SEQ ID NO: 10, SEQ ID NO: 11, SEQ ID NO: 15, SEQ ID NO: 16, SEQ ID NO: 19 and a fragment of at least 30 nucleotides thereof, and optionally SEQ ID NO: 22 or a fragment of at least 30 nucleotides thereof;

SEQ ID NO: 1, SEQ ID NO: 2, SEQ ID NO: 3, SEQ ID NO: 4, SEQ ID NO: 5, SEQ ID NO: 10, SEQ ID NO: 14, SEQ ID NO: 15, SEQ ID NO: 16, SEQ ID NO: 19 and a fragment of at least 30 nucleotides thereof;

SEQ ID NO: 1, SEQ ID NO: 2, SEQ ID NO: 3, SEQ ID NO: 10, SEQ ID NO: 11, SEQ ID NO: 14, SEQ ID NO: 15, SEQ ID NO: 16, SEQ ID NO: 17, SEQ ID NO: 19 and a fragment of at least 30 nucleotides thereof, and optionally SEQ ID NO: 22 and/or SEQ ID NO: 23 and/or SEQ ID NO: 24 or a fragment of at least 30 nucleotides thereof;

SEQ ID NO: 1, SEQ ID NO: 2, SEQ ID NO: 3, SEQ ID NO: 10, SEQ ID NO: 15, SEQ ID NO: 16, SEQ ID NO: 17, SEQ ID NO: 19 and a fragment of at least 30 nucleotides thereof;

SEQ ID NO: 1, SEQ ID NO: 2, SEQ ID NO: 3, SEQ ID NO: 4, SEQ ID NO: 5, SEQ ID NO: 10, SEQ ID NO: 11, SEQ ID NO: 14, SEQ ID NO: 15, SEQ ID NO: 16, SEQ ID NO: 17, SEQ ID NO: 19 and a fragment of at least 30 nucleotides thereof, and optionally SEQ ID NOs: 22, 23 and 24 or a fragment of at least 30 nucleotides thereof; and;

SEQ ID NO: 1, SEQ ID NO: 2, SEQ ID NO: 3, SEQ ID NO: 10, SEQ ID NO: 14, SEQ ID NO: 15, SEQ ID NO: 16, SEQ ID NO: 19 and a fragment of at least 30 nucleotides thereof.

In a very specific aspect, the combination comprises or consists of any one of the following combinations of biomarkers:

SEQ ID NO: 1, SEQ ID NO: 2, SEQ ID NO: 3, SEQ ID NO: 4, SEQ ID NO: 10, SEQ ID NO: 11, SEQ ID NO: 15, SEQ ID NO: 16, SEQ ID NO: 19, and SEQ ID NO: 22 or a fragment of at least 30 nucleotides thereof;

SEQ ID NO: 1, SEQ ID NO: 2, SEQ ID NO: 3, SEQ ID NO: 4, SEQ ID NO: 5, SEQ ID NO: 10, SEQ ID NO: 14, SEQ ID NO: 15, SEQ ID NO: 16, and SEQ ID NO: 19 or a fragment of at least 30 nucleotides thereof;

SEQ ID NO: 1, SEQ ID NO: 2, SEQ ID NO: 3, SEQ ID NO: 10, SEQ ID NO: 11, SEQ ID NO: 14, SEQ ID NO: 15, SEQ ID NO: 16, SEQ ID NO: 17, SEQ ID NO: 19, SEQ ID NO: 22, SEQ ID NO: 23 and SEQ ID NO: 24 or a fragment of at least 30 nucleotides thereof; and

SEQ ID NO: 1, SEQ ID NO: 2, SEQ ID NO: 3, SEQ ID NO: 10, SEQ ID NO: 11, SEQ ID NO: 14, SEQ ID NO: 15, SEQ ID NO: 16, SEQ ID NO: 17 and SEQ ID NO: 19 or a fragment of at least 30 nucleotides thereof.

In one embodiment, the invention relates to the use of at least nine biomarkers used in combination and wherein said combination comprises or consists in biomarkers of SEQ ID NO: 1, SEQ ID NO: 2, SEQ ID NO: 3, SEQ ID NO: 10, SEQ ID NO: 11, SEQ ID NO: 14, SEQ ID NO: 15, SEQ ID NO: 16 and SEQ ID NO: 17, or fragment of at least 30 nucleotides thereof.

Preferably, the invention relates to the use of nine biomarkers of SEQ ID NO: 1, SEQ ID NO: 2, SEQ ID NO: 3, SEQ ID NO: 10, SEQ ID NO: 11, SEQ ID NO: 14, SEQ ID NO: 15, SEQ ID NO: 16 and SEQ ID NO: 17, respectively, or fragment of at least 30 nucleotides thereof.

Even more preferably, the invention relates to the use of six biomarkers of SEQ ID NO: 1, SEQ ID NO: 2, SEQ ID NO: 3, SEQ ID NO: 10, SEQ ID NO: 15, and SEQ ID NO: 16, respectively, or fragment of at least 30 nucleotides thereof.

Particularly, the lncRNA biomarkers of SEQ ID NO: 1, SEQ ID NO: 3, SEQ ID NO: 4, SEQ ID NO: 5, SEQ ID NO: 7, SEQ ID NO: 8, SEQ ID NO: 9, SEQ ID NO: 10, SEQ ID NO: 13, SEQ ID NO: 15, SEQ ID NO: 20 are lincRNA biomarkers.

Particularly, the lncRNA biomarkers of SEQ ID NO: 2, SEQ ID NO: 6, SEQ ID NO: 11, SEQ ID NO: 12, SEQ ID NO: 14, SEQ ID NO: 16, SEQ ID NO: 17, SEQ ID NO: 18, SEQ ID NO: 19, SEQ ID NO: 21, SEQ ID NO: 22, SEQ ID NO: 23, SEQ ID NO: 24 are antisense lncRNA biomarkers.

Preferably, the above mentioned biomarker(s) is/are further used in combination with additional prostate cancer biomarkers or fragments thereof, preferably such biomarkers being lncRNAs prostate cancer biomarkers or fragments thereof, more preferably such biomarkers being the PCA3 antisense lncRNA (e.g. Gene ID: 50652 or GenBank: AF103907.1) or a fragment thereof, even more preferably the biomarker of SEQ ID NO 19 or a fragment of at least 30 nucleotides thereof.

In a particular embodiment, the above mentioned use or method relate to a combination of no more than 20 lncRNAs biomarkers or fragments thereof, preferably no more than 15 lncRNAs biomarkers, even more preferably no more than 10 lncRNAs biomarkers.

Preferably, the above mentioned lncRNA biomarkers are specifically expressed by prostate cancer cells or tissue and are not naturally expressed by non-tumoral kidney cells, non-tumoral bladder cells and/or non-tumoral prostate cells.

### Additional Biomarkers

In any of the uses or methods disclosed herein, at least one additional biomarker may be provided. Such at least one additional biomarker is preferably a prostate cancer biomarker or fragments thereof, preferably lncRNAs prostate cancer biomarkers or fragments thereof.

Non-limiting other examples of prostate cancer markers include PSA and other kallikrein family members, GSTP1 (Glutathione S-Transferase Pi-1), AMACR (Alpha-Methyl-Acyl-CoA Racemase), ERG (ETS-Related Gene), gene fusions involving ETS (Erythroblast Transformation-Specific)-related genes, and PCGEM1 (prostate-specific transcript 1 (non-protein coding)).

Preferably, the additional biomarker is the PCA3 antisense lncRNA (e.g. Gene ID: 50652 or GenBank: AF103907.1) or a fragment thereof, even more preferably the biomarker of SEQ ID NO: 19 or a fragment of at least 30 nucleotides thereof.

The determination of the amounts of lncRNA biomarkers of the invention and other prostate cancer markers can be carried out in the same or in different reaction mixtures, simultaneously or not.

Different prostate cancer scores can also be calculated to contribute to the diagnostic of the subject: the prostate specific antigen (PSA) Score, the Myriad Prolaris Assay (MPA) Score, the Oncotype DX Genomic Prostate Score (GPS), the Cancer of the Prostate Risk Assessment (CAPRA) Score, and the Gleason Score.

### Diagnosis Method

In a second aspect, the present invention also concerns an in-vitro method for prostate cancer diagnosis of a subject, wherein the method comprises the step of determining the amount of at least one lncRNA biomarker as defined above or any combination thereof as defined above in a biological sample from said subject, and wherein an increased amount of the at least one said lncRNA biomarker or combination thereof is indicative of prostate cancer. The invention may particularly comprise the determination of the amount (or level) of one or several lncRNAs, and a correlation of said amount to the presence, absence or characteristic of prostate cancer. In some embodiments, it is desirable to simultaneously determine the expression level of a plurality of different lncRNAs in the test sample, even more preferably of a combination of lncRNAs (e.g. a lncRNA signature) for prostate cancer diagnosis purpose.

Particularly, the present invention relates to an in-vitro method for the discrimination of high- and intermediate-risk prostate tumors versus low-risk and normal tissues for the diagnosis of prostate cancer in a subject, wherein the method comprises the step of determining the amount of at least one lncRNA biomarker as defined above or any combination thereof as defined above in a biological sample from said subject, and wherein an increased amount of the at least one said lncRNA biomarker or combination thereof is indicative of prostate cancer.

The diagnosis methods disclosed herein particularly comprises a step of determining the amount of at least one lncRNA biomarker or a fragment thereof in a biological sample from the subject.

Prior to this step, the method may further comprise a step of obtaining or providing a sample from the subject.

The method may also comprise a step of preparing or extracting the nucleic acids, preferably the ribonucleic acids, from the sample. The amount of at least one lncRNA biomarker may then be quantified in the preparation of RNAs extracted from the sample.

A test value, expression level or other calculated test level of a lncRNA biomarker or fragment thereof, or other biomarker refers to an amount of a biomarker, such as an lncRNA or fragment thereof, in a subject's undiagnosed biological sample. The test level may be compared to that of a control sample, or may be analyzed based on a reference standard that has been previously established to determine a status of the sample. A test sample or test amount can be either in absolute amount (e.g., number of copies/ml, nanogram/ml or microgram/ml) or a relative amount (e.g., relative intensity of signals).

A control value, expression level or other calculated level may be any amount or range of amounts to be compared against a test amount of a biomarker. A control level may be the amount of a biomarker in a healthy or non-diseased state of a cell, a tissue or a subject. For example, a control amount of a biomarker can be the amount of a biomarker in a population of patients with a specified condition or disease or a control population of individuals without said condition or disease. The control amount of biomarker may also be the amount of the lncRNA biomarker in a corresponding healthy tissue. In the context of prostate cancer, the control amount of a biomarker may be determined in a healthy prostate tissue from the same patient. A control amount can be either in absolute amount (e.g., number of copies/ml, nanogram/ml or microgram/ml) or a relative amount (e.g., relative intensity of signals).

Particularly, the method may further comprise the determination of the amount of additional prostate cancer markers or fragments thereof, preferably lncRNAs which are prostate cancer biomarkers as described hereabove.

### Sample

The term “biological sample”, as used herein, means any sample containing lncRNAs derived from the subject. Examples of such biological samples include fluids such as blood, plasma, urine, seminal fluid samples or mixed urine and seminal fluid (first urine sample following ejaculation) as well as biopsies, organs, tissues or cell samples, in particular a prostate sample. Preferably, the biological sample is a blood or a urine sample. More preferably, the biological sample is a urine sample.

In one embodiment of the above mentioned method, the sample, preferably a urine sample, is obtained after an attentive digital-rectal examination (DRE) and/or a prostate specific antigen (PSA) test, preferably a urine sample is obtained if the PSA test and/or the digital-rectal examination were indicative of prostate cancer.

In a particular embodiment of the above mentioned method, the urine sample is obtained just after a digital-rectal examination. Indeed, the digital-rectal examination increases the amount of prostatic cells and prostatic cell fragments in the urine sample, thereby facilitating the detection of the lncRNAs.

Of course, it should be understood that the present method can also be used on a sample obtained without being preceded by a digital-rectal examination and/or PSA test.

A urine preservative can be added to the sample, preferably a Norgen tubes preservative (ref. 18113 Norgen bioteck corporation), allowing the conservation of the sample at room temperature for 2 years.

LncRNAs may be extracted or partially purified from a biological sample. To that purpose, total RNAs may be purified by homogenization in the presence of a nucleic acid extraction buffer, followed by centrifugation. RNA molecules may be separated by electrophoresis on agarose gel(s) following standard techniques.

### Nucleic Acid Extraction

Several methods and kits are available for the person skilled in the art to extract the nucleic acids, and more particularly the ribonucleic acids, contained in the sample. For example, extraction may rely on lytic enzymes or chemical solutions or can be done with nucleic-acid-binding resins following the manufacturer's instructions.

In one embodiment of the above mentioned method, the cells, cell fragments and exosomes present in the sample, preferably a urine sample, are collected, preferably by centrifugation. A total nucleic acid extraction is then carried out. Preferably, the lncRNAs biomarker nucleic acids are extracted from prostate cells or from a human urine sample.

Non-limiting example are a phenol/chloroform or Trizol extraction methods. Total nucleic acid extraction may also be carried out using a solid phase band method on silica beads. Of course, it should be understood that numerous nucleic acid extraction methods exist and thus, that other methods can be used in accordance with the present invention.

As used herein, the term “exosome” refers to any kind of cell-derived vesicles present in biological fluids, including blood and urine. This term encompasses “microvesicles”, “epididimosomes”, “argosomes”, “exosome-like vesicles”, “microparticles”, “promininosomes”, “prostasomes”, “dexosomes”, “texosomes”, “dex”, “tex”, “archeosomes” “oncosomes”, “liposomes” and “micelles”.

Preferably, after total nucleic acid extraction, DNA is degraded so as to conserve only the RNA molecules. In particular, a deoxyribonuclease may be used to degrade DNA.

A variety of nucleic acids quantification techniques, well known by the skilled person, can be used to determine the amount of at least one lncRNA biomarker from a biological sample, and in particular from the RNAs extracted from said sample. These techniques can be adapted in accordance with the type of sample, the sensitivity of the quantification desired, the amount of nucleic acid in the sample, and the like.

In particular, measurement of the amount of at least one lncRNA biomarker can be direct or indirect. Indeed, the amount of an lncRNA biomarkers can be directly quantified, preferably by hybridization, more preferably by hybridization of a labeled specific probe, still more preferably by hybridization of a fluorescent labeled specific probe immobilized directly or indirectly on a solid support, even more preferably by the Nanostring method.

Particularly, the method for determining the amount of at least one lncRNA biomarker may involve probes, in particular labeled probes.

The term “label”, as used herein, refers to any atom or molecule that can be used to provide a quantifiable signal and that can be attached to a nucleic acid via a covalent bond or noncovalent interaction (e.g., through ionic or hydrogen bonding, or via immobilization, adsorption, or the like).

The probes of the present invention can be labeled by standard labeling techniques such as with a radiolabel, enzyme label, fluorescent label, biotin-avidin label, chemiluminescent label, and the like. After hybridization, the probes can be visualized using known methods. In particular, labels generally provide signals detectable by fluorescence, chemiluminescence, radioactivity, colorimetry, mass spectrometry, X-ray diffraction or absorption, magnetism, enzymatic activity, or the like.

Preferably, the detectable label may be a luminescent label. For example, fluorescent labels, bioluminescent labels, chemiluminescent labels, and colorimetric labels may be used in the practice of the invention, more preferably a fluorescent label.

The terms “fluorescent label”, “fluorophore”, “fluorogenic dye”, “fluorescent dye” as used herein are interchangeable and designate a functional group attached to a nucleic acid that will absorb energy of a specific wavelength and re-emit energy at a different, but equally specific, wavelength.

Fluorescent labels that can be used in the context of this invention include, but are not limited to, fluorescein, a phosphor, a rhodamine, or a polymethine dye derivative.

Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. The fluorescent label can be made of a combination of fluorescent labels.

In one embodiment of the above described method, the probe is immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

In a particular embodiment, the amount of an lncRNA biomarker can be determined indirectly, especially after its conversion to cDNA, preferably by amplification.

Methods of amplification include ligase chain reaction (LCR), transcription-mediated amplification (TMA), strand displacement amplification (SDA), nucleic acid sequence based amplification (NASBA), microarray analysis, ChTP, serial analysis of gene expression (SAGE), next-generation RNA sequencing (e.g., deep sequencing, whole transcriptome sequencing, exome sequencing), gene expression analysis by massively parallel signature sequencing (MPSS), immune-derived colorimetric assays, situ hybridization (ISH) formulations (colorimetric/radiometric) that allow histopathology analysis, mass spectrometry (MS) methods, RNA pull-down and chromatin isolation by RNA purification (ChiRP), and proteomics-based identification (e.g., protein array, immunoprecipitation) of lncRNA. These methods are well known by the skilled person in the art.

In a particular embodiment, the amount of an lncRNA biomarker is determined indirectly, especially after its conversion to cDNA, preferably by amplification, especially a quantitative amplification, more preferably by an amplification method coupled to real-time detection of the amplified products, even more preferably by quantitative RT-PCR.

### Quantitative RT-PCR

In one embodiment of the above mentioned method, the amount of at least one lncRNA biomarker is quantified using an RNA reverse-transcription and amplification method. In such an embodiment, the RNA amplification method is coupled to a quantitative amplification such as real-time detection of the amplified products (e.g. issued from the targeted lncRNA biomarker or fragment thereof) using fluorescence specific probes. Such probes target specifically (e.g. specifically hybridizes with) a lncRNA biomarker or fragment thereof to reveal and/or quantify the presence or amount of the lncRNA biomarker in a sample. The amount of lncRNAs in a sample can also be determined by specific amplification. Various techniques exist for amplifying lncRNAs nucleic acid sequences, including without limitation reverse transcription (RT), polymerase chain reaction (PCR), real-time PCR (quantitative PCR (q-PCR)), nucleic acid sequence-base amplification, multiplex ligatable probe amplification, rolling circle amplification, or strand displacement amplification. In a preferred embodiment, the determination is made by reverse transcription of lncRNAs, followed by amplification of the reverse-transcribed transcripts by polymerase chain reaction (RT-PCR). Amplification generally uses nucleic acid primers.

Preferably, the amplification method is PCR. More preferably, the PCR is a quantitative PCR or a related method enabling detection in real-time of the amplified products.

Among the amplification methods, quantitative reverse transcription PCR (quantitative RT-PCR) is preferred.

As used herein, the terms “quantitative RT-PCR”, “qRT-PCR”, “Real time RT-PCR” and “quantitative Real time RT-PCR” are equivalent and can be used interchangeably.

Likewise, the terms “quantitative PCR”, “qPCR”, “Real time PCR” and “quantitative real time PCR” are equivalent and can be used interchangeably.

Any of a variety of published quantitative RT-PCR protocols can be used (and modified as needed) for use in the present method. Suitable quantitative RT-PCR procedures include but are not limited to those presented in U.S. Pat. No. 5,618,703 and in U.S. Patent Application No. 2005/0048542, which are hereby incorporated by reference.

In a preferred embodiment of the above mentioned method, the quantitative RT-PCR includes two main steps, the reverse transcription (RT) of RNA in cDNA and the quantitative PCR (Polymerase Chain Reaction) amplification of the cDNA.

Quantitative RT-PCR can be performed by an uncoupled or by a coupled procedure. In an uncoupled quantitative RT-PCR, the reverse transcription is performed independently from the quantitative PCR amplification, in separate reactions. Whereas, in a coupled quantitative RT-PCR, the reverse transcription and the quantitative PCR amplification are performed in a single reaction tube using a common reaction mixture including both the reverse transcriptase and the DNA polymerase. The method of the invention encompasses all versions of quantitative RT-PCR.

The term “reaction mixture” or “master mix” or “master mixture” refers to an aqueous solution of constituents in a quantitative RT-PCR reaction that can be constant across different reactions. In case of a coupled procedure, an exemplary “quantitative RT-PCR reaction mixture” includes buffer, a mixture of deoxyribonucleoside triphosphates, reverse transcriptase, RT and PCR primers, probes, and DNA polymerase. In case of uncoupled procedure, two reaction mixtures are needed, a “RT reaction mixture” including, for example, buffer, a mixture of deoxyribonucleoside triphosphates, reverse transcriptase and RT primers, and a “quantitative PCR” or “PCR reaction mixture” including, for example, buffer, a mixture of deoxyribonucleoside triphosphates, PCR primers, probes, and DNA polymerase.

The RNA or cDNA templates (i.e. comprising the lncRNA biomarker(s) or fragment thereof) have to be added to theses reaction mixtures.

### Reverse Transcription

The reverse transcription includes three basic steps: (1) denaturation of RNA, (2) hybridizing of the RT primers, (3) synthesis of cDNA.

The term “RT primers”, as used herein, mean oligonucleotide primers that anneal to RNA and allow reverse transcriptase to elongate it into cDNA. Two different types of RT primers may be used, non-specific oligonucleotide RT primers or sequence specific oligonucleotide RT primers.

Using non-specific oligonucleotide RT primers such as Oligo dT (specific of polyA tail of RNAs) or random primers (for example hexamers) allows the reverse transcription of the most of the RNA present in the sample.

Using sequence specific RT primers (primers complementary to a sequence of the target RNA) allows the reverse-transcription of only the RNAs of interest. In particular, a sequence specific RT primer can be a primer which has a nucleic acid sequence complementary to the antisense lncRNA target but not to the corresponding mRNA, allowing the specific reverse transcription of the antisense lncRNA. However, to conduct a reverse transcription with a sequence specific RT primer, a multiplexing (use of several primers targeting several distinct RNA molecules) or multiple RT reactions are needed. In the latter case a greater amount of RNA is needed.

Preferably, the reverse transcription is non-specific, more preferably the RT primers used are random primers.

Reverse transcriptase enzymatic activity provides a cDNA transcript from an RNA template. There are many reverse transcriptases that are commercially available (e.g., Moloney Murine Leukemia Virus (M-MuLV) Reverse Transcriptase from New England Biolabs, Inc., Beverly, Mass.; HIV reverse transcriptase from Ambion, Inc., Austin, Tex.). The methods of the invention are not limited to any particular enzyme, although some enzymes may be preferable under specific conditions. The two most commonly used reverse transcriptases are avilo myeloblastosis virus reverse transcriptase (AMV-RT) and Moloney murine leukemia virus reverse transcriptase (MLV-RT).

Prior to the quantitative amplification by PCR, in case of an uncoupled procedure:
the reverse transcription may further comprises a step of RNA degradation, for example by addition of an RNA nuclease to the reaction mixture; and/or
the reverse transcription may further comprise a step of inactivating the reverse transcriptase.

### Quantitative PCR

Quantitative PCR allows quantification of reaction products for each sample per cycle. Commonly used instrumentation and software products perform the quantification calculations automatically. The PCR process generally consists in the repetition of a sequence of temperature changes (or cycle) conducted by a thermal cycler. Usually, 25 to 50 cycles are needed.

These cycles normally consist of three stages: the first (denaturation), at around 95° C., allows the separation of the nucleic acids double chain; the second (alignment), at a temperature of around 50-60° C., allows the binding of the PCR primers with the DNA template; the third (elongation), at between 68-72° C., facilitates the polymerization carried out by the DNA polymerase. Due to the small size of the fragments amplified in quantitative PCR, the last step is usually omitted as the enzyme is able to increase their number during the change between the alignment stage and the denaturing stage. Preferably, a step of fluorescence measurement may be added. The temperatures and the timings used for each cycle depend on a wide variety of parameters, such as the DNA polymerase used, the concentration of divalent ions and deoxyribonucleotides in the reaction mixture and the binding temperature of the primers.

The recent advancement in PCR instrumentation technology (e.g., Cepheid's Smart Cycler® II), allows the simultaneous detection and quantification of different fluorescent signals in different channels (PCR multiplex) in real-time. In addition, the latest generation of thermal cyclers are designed to maximize fluorescent dye excitation providing a more accurate means of detecting fluorescence. Thus, multiple amplification products can be assessed in the same reaction mixture and quantified more accurately. For example, the amounts of at least 2, at least 3, at least 4 at least 5, at least 6, at least 7 at least 8, at least 9, at least 10, at least 11, at least 12, at least 13, at least 14, at least 15, at least 16, at least 17, at least 18, or at least 19 lncRNAs biomarkers as defined above (i.e. of SEQ ID NOs: 1-19 or fragment thereof) can be simultaneously determined by quantitative RT-PCR.

The term “PCR primer” or “primer pair”, as used herein, are equivalent and mean a pair of oligonucleotide primers that are complementary to the sequences within a target cDNA sequence in a PCR. The primer pair consists of a forward primer and a reverse primer which nucleic acid sequences are complementary to a minus (reverse) and a plus (forward) strand of the double stranded cDNA fragment of interest, respectively.

Primers of a primer pair should have similar hybridizing conditions and in particular similar melting temperatures since annealing in a PCR occurs for both simultaneously. A primer with a Tm (melting temperature) significantly higher than the reaction's annealing temperature may mishybridize and extend at an incorrect location along the DNA sequence, while a primer with a Tm significantly lower than the annealing temperature may fail to anneal and will not extend at all. Primer sequences also need to be chosen to uniquely select for a region of DNA, avoiding the possibility of mishybridization to a similar sequence nearby.

It is not necessary that every nucleotide of the PCR primers anneal to the template to allow the amplification of the cDNA. For example, a non-complementary nucleotide fragment may be attached to the 5′ end of the PCR primers with the remainder of the primer sequence being complementary to the cDNA. Alternatively, non-complementary bases can be interspersed into the PCR primer, provided that the primer sequence has sufficient complementarity with the cDNA. Thus, the embodiments of the invention contemplate variants of the PCR primers described herein.

In one particular embodiment of the above mentioned method, the primers span the 3′ region of a first exon and the 5′ region of a second exon, so as such primers can only amplify sequences which the first and second exon have been spliced into a contiguous position (i.e. by removing an intervening intronic sequence). Knowing the sequences of the exon boundaries, as well as those of the different exons, the primers which can be designed and used in the context of the present invention can be readily determined by a person of ordinary skill in the art to which the present invention pertains.

In a preferred embodiment of the above mentioned method, a primer pair consists in primers that are complementary to exon sequences of the lncRNA, but are obligatory out of exons of the corresponding sense-paired pre-mRNA in case of the said lncRNA is the antisense lncRNA. Such primers cannot hybridize to the cDNA reversed transcript from said mRNA or any sequence deriving from it. Preferably, the primers are complementary to exon sequences, even more preferably to two adjacent exon sequences of the lncRNA and are obligatory out of exons of the corresponding sense paired pre-mRNA.

This primer pair allows the specific amplification of the cDNA reversed transcript from the lncRNA biomarker or fragment thereof.

Many DNA polymerases suitable for quantitative PCR are commercially available (e.g., Taq and T7 DNA polymerases from New England Biolabs, Inc.; Pfu DNA polymerase from Promega, Inc., Madison, Wis.). The method of the invention is not limited to any particular enzyme, although some enzymes may be preferable under specific conditions.

The term “probe”, in the context of the quantitative RT-PCR, refers to an oligonucleotide that hybridizes to a target sequence situated between the annealing sites of the two primers of the primer pair. The probe includes a detectable label, e.g., a fluorophore (Texas-Red®, Fluorescein isothiocyanate, etc.) that can be covalently attached directly to the probe oligonucleotide, e.g., located at the probe's 5′ end or at the probe's 3′ end. The probe may also include a quencher. A probe includes about 6 nucleotides, about 8 nucleotides, about 10 nucleotides, about 15 nucleotides, about 20 nucleotides, about 30 nucleotides, about 40 nucleotides, or about 50 nucleotides. In some embodiments, a probe includes from about 6 nucleotides to about 40 nucleotides. Particularly, the probe specifically target at least one lncRNA biomarker or fragment thereof.

The term “quenching”, as used herein, refers to a decrease in fluorescence of a fluorescent detectable label caused by energy transfer associated with a quencher moiety, regardless of the mechanism. Suitable quencher moiety is for example Black Hole Quencher™ (Biosearch Technologies, Novato, Calif.) and Iowa Black (Integrated DNA Technologies, Coralville, Iowa).

Four different probe systems are in current use for quantitative PCR-Molecular Beacons (Sigma-Genosys, Inc., The Woodlands, Tex.), Scorpions® (DxS Ltd., Manchester, UK), SYBR® Green (Molecular Probes, Eugene, Oreg.), and TaqMan® (Applied Biosystems, Foster City, Calif.). These four systems employ fluorescent labels that the instrumentation detects and the software interprets levels of fluorescence.

SYBR® Green is a fluorescent dye that only strongly fluoresces when bound to double stranded DNA. SYBR® green assay is a quick method, particularly useful for detection.

Molecular Beacons, Scorpions®, and TaqMan® utilize Förster Resonance Energy Transfer (FRET) by coupling a fluorescent label with a quencher moiety. A fluorescent label is covalently bound to the 5′ end of an oligonucleotide probe, while the 3′ end has a quencher moiety attached. These oligonucleotide probes are site specific to hybridize to the amplified product. Preferably, the oligonucleotide probes are designed to hybridize to a central region of the amplified product.

For TaqMan® assays, the 5′→3′ exonuclease activity of the DNA polymerase cleaves the probe during the elongation cycle. Due to the cleavage of the probe, the quencher moiety is no longer coupled to the fluorescence label and cannot quench fluorescence. One molecule of reporter fluorescent dye is liberated for each new molecule synthesized, and detection of the unquenched reporter fluorescent dye provides the basis for quantitative interpretation of the data. Fluorescence thus represents replicating DNA.

Similarly, Molecular Beacons utilizes an oligonucleotide probe with a fluorescent label attached to the 5′ end and a quencher moiety attached to the 3′ end. When free in solution, the Molecular Beacons oligonucleotide probe forms a hairpin structure. In the hairpin structure, the quencher moiety is able to quench fluorescence due to FRET. However, during PCR, the oligonucleotide probe unfolds and hybridizes to its complementary DNA, and the quencher is no longer close enough to the fluorescent label to quench fluorescence. Thus, fluorescence reports the hybridization between an oligonucleotide probe and its specific target cDNA.

Scorpions® also utilize an oligonucleotide probe with a fluorescent label attached to the 5′ end and a quencher moiety attached to the 3′ end in a hairpin structure free in solution. However, the Scorpions® oligonucleotide probe also serves as a primer. The Scorpions® oligonucleotide probe/primer extends from the hairpin loop structure and hybridizes to the target. The Scorpions® oligonucleotide probe/primer fluoresces, and the DNA polymerase extends the target DNA from the primer. Thus, the probe detects the extension product, which is its own primer-unimolecular rearrangement. Thereby, the fluorescence reports the extension and thus copy number of reaction product.

In a preferred embodiment, the present method employs a SYBR® Green fluorescent dye. Alternatively, the present method employs TaqMan®-style probes, i.e. dual-labeled probes aimed to fluoresce upon 5′→3′ exonuclease activity.

### Analysis of the Quantitative RT-PCR Results

RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). The system consists of a thermocycler, laser, charge-coupled device (CCD), camera and computer. The system includes software for running the instrument and for analyzing the data (determining the quantity of amplification product based upon the fluorescence data).

To minimize errors and the effect of sample-to-sample variations, quantitative RT-PCR is usually performed using an internal reference. The ideal internal reference is expressed at a constant level among different samples, and is unaffected by the experimental treatment. RNAs most frequently used to normalize patterns of gene expression are mRNAs for the housekeeping genes glyceraldehyde-3-phosphate-dehydrogenase (GAPDH) and beta-actin.

A standard curve can be generated from a cDNA of known concentration. The standard curve can then be used to determine absolute or relative cDNA levels.

The comparative cycle threshold (Ct) method, also known as the 2-ΔΔCt method, allows to quantify cDNA levels. Fluorescence values are recorded during every cycle and represent the amount of product amplified to that point in the amplification reaction. The point when the fluorescent signal is first recorded as statistically significant is the threshold cycle (Ct).

The Ct method compares a test reaction with a control or calibrator sample. The Ct values of both the control/calibrator sample and the test sample are normalized. In an embodiment of the invention, the Ct values may be normalized to an arbitrary cutoff (e.g. 20-22).

The Ct method can also be described by the formula ΔΔCt=ΔCttest sample−ΔCt reference sample. The amplification efficiencies of the test sample and the reference sample must be about the same for the formula to operate. Amplification efficiencies can be determined by a comparison of the samples with template dilution. The amplification efficiency is about the same when a plot of cDNA dilution versus ΔCt approximates zero.

In some embodiments, the test level and the control level may be expressed as a mean comparative quantification (Cq) test value and a mean comparative quantification (Cq) control value (delta Cq method). In such a case, the mean Cq test value and a mean Cq control value are normalized by an internal control. For example, in tumor tissue samples, the difference of threshold cycle (Cq) values obtained for the target lncRNA (e.g., of SEQ ID NO: 1) and internal control in a cancer specimen can be compared to the difference of the Cq values obtained in adjacent normal tissue. The delta-delta Cq method may then be used to calculate the relative expression values between tissue samples.

In one embodiment of the above mentioned method, the amount of at least one lncRNA or antisense lncRNA is determined by quantitative RT-PCR, wherein said quantitative RT-PCR comprises the steps of:
reverse transcription of the RNAs present in the sample into cDNAs; and
amplification of the cDNA reverse transcript from the at least one lncRNA or a part or fragment of said cDNA by quantitative PCR, wherein said part or fragment has a length of at least 30 nucleotides.

Preferably, said reverse transcription comprises a step of contacting said RNAs with RT primers, more preferably random primers.

Preferably, said amplification comprises a step of contacting said cDNA with at least one primer pair, more preferably said amplification further comprises the step of contacting said cDNA with at least one labeled probe. The labeled probe of the invention is complementary to exon sequences of the lncRNA, but is obligatory out of exons of the corresponding sense paired pre-mRNA, if the said lncRNA is the antisense lncRNA. More preferably, the labeled probe is complementary to two adjacent exon sequences of the lncRNA and is obligatory out of exons of the corresponding pre-mRNA. Thus, the labeled probe of the invention is able to label the lncRNA but not the corresponding sense pre-mRNA.

Preferably, the at least one primer pair consists in primers that are complementary to exon sequences of the lncRNA, but are obligatory out of exons of the corresponding sense paired pre-mRNA. More preferably, the primers are complementary to two adjacent exon sequences of the lncRNA and are obligatory out of exons of the corresponding pre-mRNA. Thus, primer pairs of the invention are able to amplify the lncRNA but not the corresponding sense pre-mRNA.

Preferably, the amplification further comprises a step of contacting said cDNAs with a SYBR® Green fluorescent dye, alternatively said amplification further comprises a step of contacting said cDNAs with at least a dual labeled probe, i.e. a probe with a fluorescent label and a quencher moiety, said at least one dual labeled probe being complementary to a sequence of cDNA localized between the two primers of the primer pair. More preferably, the dual labeled probe fluoresce upon 5′→3′ exonuclease activity.

Preferably, when said at least one labeled probe comprises more than one probe, each probe has a different label, i.e. fluoresces at a different wavelength.

Alternatively, a combination of two different primer pairs specific of the same lncRNA biomarker are used to amplify the cDNA by quantitative PCR.

In a particular embodiment of the above mentioned method, the amount of lncRNA biomarkers is determined by quantitative RT-PCR, wherein said quantitative RT-PCR comprises the steps of:
reverse transcription of the RNAs present in the sample into cDNAs,
amplification of the cDNA reverse transcripts from the lncRNAs or a part of said cDNAs by quantitative PCR, wherein said part or fragment has a length of at least 30 nucleotides and wherein said amplification comprises the step of contacting said cDNAs with primer pairs. Preferably, each primer pair is specific to one lncRNA biomarker or fragment thereof.

Preferably, said amplification further comprises the step of contacting said cDNAs with labeled probes, more preferably dual labeled probes, even more preferably dual labeled probe that fluoresce upon 5′→3′ exonuclease activity, wherein each of said dual labeled probes are complementary to a sequence of a cDNA localized between the two primers of the primer pairs.

Hybridization Method

In one embodiment of the above mentioned method, the amount of at least one lncRNA or antisense lncRNA is determined using an RNA hybridization method. In such an embodiment, the RNA hybridization method is coupled to the detection of the hybridized RNA using a probe, preferably a labeled specific probe. Even more preferably, the specific probe is labeled by a fluorophore.

When the amounts of several lncRNA biomarkers are determined simultaneously, each probe is linked to a different label. Preferably, each probe is specific of one lncRNA biomarker or fragment thereof.

The complex probe/lncRNA biomarker may be bound on a solid support, allowing to wash the reaction of the unbound probes.

After extraction of the RNA from the sample, the determination of the amount of at least one lncRNA biomarker by an RNA hybridization method comprises the following steps:
Contacting at least one labeled specific probe with the extracted RNAs in conditions suitable for hybridization of said probe with its lncRNA biomarker target; and
Quantification of the signal emitted by the probe.

Optionally, the RNAs are denatured prior to the contacting step, thereby removing the RNA secondary structure.

In a preferred embodiment, the at least one lncRNA biomarker is immobilized on a solid support. Optionally, after immobilization of the at least one lncRNA biomarker and the step of contacting, the method described above comprises a washing step, thereby removing the unbound probes.

The lncRNA biomarkers may be directly immobilized on a solid support prior to be contacted with the probes. In such an embodiment, the amount of at least one lncRNA biomarker is preferably determined by Northern blot.

Alternatively, the lncRNA biomarkers may be indirectly immobilized on a solid support through its hybridization to its specific probe. Particularly, the sample may be contacted with probes already immobilized on a solid support. Preferably, the complex lncRNA biomarker/probe is immobilized on a solid support after hybridization.

Preferably, the probe is linked to a molecule that helps the probe to bind a solid support. More preferably said molecule is biotin, thereby the probe and its complementary lncRNA are immobilized on the solid support.

Examples of solid supports suitable for the invention include, but are not limited to, plastics such as polycarbonate, complex carbohydrates such as agarose and sepharose, and acrylic resins such as polyacrylamide and latex beads. Techniques for coupling nucleic acid and/or nucleic acid probes to such solid supports are well known in the art.

In another particular embodiment, the amount of at least one lncRNA biomarker is determined by a LNA (Lock nucleic acid) method (see for example the techniques developed by Exiqon).

In a most preferred embodiment, the amount of at least one lncRNA biomarker is determined by the Nanostring method.

### Nanostring Method

The Nanostring method is a hybridization method that allows to quantify RNA without requiring linear (array) nor exponential (PCR) amplification. It is a very sensitive method, since only 10 ng of RNA are needed to perform it, allowing analysis of quantity limited biological samples, such as urine. The restricted number of sample manipulation steps together with the absence of enzymatic reaction allows precise and physiologically correct quantifications. This method is also extremely flexible since it can be applied to various types of samples.

The Nanostring method necessitates the use of a pair of probes specifically designed for each targeted lncRNA biomarker or fragment thereof.

The first probe, called the capture-probe, specifically hybridizes the lncRNA biomarker target and binds it to a solid support, preferably a counting stand. Preferably, the capture probe is linked to a molecule that allows the probe to bind the solid support, more preferably said molecule is biotin, thereby immobilizing the targeted lncRNA biomarker onto the counting stand.

The second probe, called the reporter-probe, specifically hybridizes the lncRNA biomarker and is linked to a label that allows the detection and quantification of the lncRNA biomarker. Preferably, this label is a fluorescent label. More preferably the label is made of a combination of fluorochromes. Even more preferably the label is made of a combination of 6 fluorochromes chosen among 4 fluorochromes of different colors, defining a code specific to each target lncRNA biomarker. This color code confers to the technique a very high sensitivity and enables the analysis of quantity-limited biological samples. When several reporter probes are used simultaneously to determine the amounts of several lncRNA biomarker, each reporter probe is linked to a different label, preferably a different combination of 6 fluorochromes chosen among 4 fluorochromes of different colors.

Preferably, the fluorescence is analyzed by an nCounter, an optical system that is capable to identify the color codes.

Preferably, the determination of the amount of at least one lncRNA biomarker by the Nanostring method comprises the following steps:
Contacting the sample with the capture and the reporter probes, in conditions suitable for hybridization with its lncRNA biomarker target; and
Elimination of the excess probes,
Alignment and immobilization of the probes/target complexes into the solid support,
Collection of the signal by the Nanostring device.

More preferably, the determination of the amount of at least one lncRNA biomarker by the Nanostring method comprises the following steps:
Contacting the sample with the capture and the reporter probes, in conditions suitable for hybridization with its lncRNA target; and
Elimination of the excess probes is achieved on a Nanostring Prep Station following a high sensitivity mode containing a dual purification process. The Nanostring prep station is robotic liquid handling system dedicated to purification of molecules coupled to probes and specially to capture probes and reporter probes. As capture probes and reporter probes contain also a specific tail (generic and specific sequence to the capture probe, idem for the reporter probes), two types of dedicated magnetic beads supplied by Nanostring will be used in this main step. First, magnetic bead coupled with sequences complementary to tails of capture probes are used to purify capture probes hybridized or not to targets of interest. After washes, purified molecules are mixed with another magnetic beads that are coupled to sequences complementary to reporter probes tails. This step selects molecules bound to the set of capture probes and reporter probes. Washes are applied to by the Nanostring Prep station to ensure correct purification of hybrids.
Next, dually purified molecules are injected in a solid device: a 12 slot Nanostring Cartridge. This device contains 12 slots meaning that 12 samples can be analyzed in the meantime. Per slot, purified and injected molecules are immobilized thanks to a streptavidin coating. A magnetic fields is also applied to align the probes/target complexes into the solid device.
Collection of the signal, in particular by the Digital Analyzer on the sample Cartridges. Color codes on the surface of the cartridge are counted and tabulated for each target molecule.

Optionally, RNAs are denatured prior to the contacting step, thereby removing the RNA secondary structure.

The Nanostring probes hybridize to their lncRNA biomarker target with a part of their nucleotide sequence which is of a length of between about 40 and about 60 nucleotides, preferably of a length of about 30 nucleotides. The capture probe may further comprise a sequence that allows to link it to biotin. The reporter probe may further comprise a sequence that allows to link it to a six fluorochromes code. The color code is specific to each lot of production done by Nanostring. To deconvoluate signals and assign color code to targets, Nanostring provides a correspondence file specific to each kit.

Preferably, the sequences of the Nanostring probes of a pair of probes target different parts of their targeted lncRNA biomarker and these parts do not overlap.

Optionally, the sequences of the Nanostring probes of a pair of probes may be chosen so as they are not distant of more than 5 nucleotides on their lncRNA biomarker target, preferably they are not distant of more than 10, 15, 20, 30, 40, 50, 100 nucleotides. In a most preferred embodiment, the Nanostring probes of a pair of probes are directly consecutive.

In one embodiment of the above mentioned method, the amount of at least one lncRNA biomarker is determined by the Nanostring method, wherein said Nanostring method comprises the step of contacting the sample with at least one capture-probe and one reporter probe, wherein said capture probe is able to specifically hybridize the at least one lncRNA biomarker and is preferably linked to a molecule that helps to immobilize said at least one lncRNA biomarker onto a solid support, more preferably said molecule is biotin, and wherein said reporter-probe is able to specifically hybridize the at least one lncRNA biomarker and is linked to a fluorescent label, preferably a combination of fluorophores, more preferably a combination of 6 fluorochromes chosen among 4 fluorochromes of different colors defining a code specific to said at least one lncRNA biomarker.

In a preferred embodiment of the above mentioned method, the amount of lncRNA biomarkers are determined by the Nanostring method, wherein said Nanostring method comprises the step of contacting the sample with a combination of lncRNA biomarkers are determined by the Nanostring method, wherein said Nanostring method comprises the step of contacting the sample with a combination pairs of probes. The person skilled in the art knows how to design such probes. In a particular embodiment, said at least one probe are selected from the group consisting of:
the probe of SEQ ID No. 25, wherein said probe is complementary to the lncRNA of SEQ ID No. 1;
the probe of SEQ ID No. 26, wherein said probe is complementary to the lncRNA of SEQ ID No. 2;
the probe of SEQ ID No. 27, wherein said probe is complementary to the lncRNA of SEQ ID No. 3;
the probe of SEQ ID No. 28, wherein said probe is complementary to the lncRNA of SEQ ID No. 4;
the probe of SEQ ID No. 29, wherein said probe is complementary to the lncRNA of SEQ ID No. 5;
the probe of SEQ ID No. 30, wherein said probe is complementary to the lncRNA of SEQ ID No. 6;
the probe of SEQ ID No. 31, wherein said probe is complementary to the lncRNA of SEQ ID No. 7;
the probe of SEQ ID No. 32, wherein said probe is complementary to the lncRNA of SEQ ID No. 8;
the probe of SEQ ID No. 33, wherein said probe is complementary to the lncRNA of SEQ ID No. 9;
the probe of SEQ ID No. 34, wherein said probe is complementary to the lncRNA of SEQ ID No. 10;
the probe of SEQ ID No. 35, wherein said probe is complementary to the lncRNA of SEQ ID No. 11;
the probe of SEQ ID No. 36, wherein said probe is complementary to the lncRNA of SEQ TD No. 12;
the probe of SEQ ID No. 37, wherein said probe is complementary to the lncRNA of SEQ ID No. 13;
the probe of SEQ ID No. 38, wherein said probe is complementary to the lncRNA of SEQ ID No. 14;
the probe of SEQ ID No. 39, wherein said probe is complementary to the lncRNA of SEQ ID No. 15;
the probe of SEQ ID No. 40, wherein said probe is complementary to the lncRNA of SEQ ID No. 16;
the probe of SEQ ID No. 41, wherein said probe is complementary to the lncRNA of SEQ ID No. 17;
the probe of SEQ ID No. 42, wherein said probe is complementary to the lncRNA of SEQ ID No. 18;
the probe of SEQ ID No. 43, wherein said probe is complementary to the lncRNA of SEQ ID No. 19;
the probe of SEQ ID No. 44, wherein said probe is complementary to the lncRNA of SEQ ID No. 20;
the probe of SEQ ID No. 45, wherein said probe is complementary to the lncRNA of SEQ ID No. 21;
the probe of SEQ ID No. 46, wherein said probe is complementary to the lncRNA of SEQ ID No. 22;
the probe of SEQ ID No. 47, wherein said probe is complementary to the lncRNA of SEQ ID No. 23; and
the probe of SEQ ID No. 48, wherein said probe is complementary to the lncRNA of SEQ ID No. 24.

Preferably, the method of the invention may use a combination of probes. Such combination may comprise or consist of the following combinations
SEQ ID NO: 25, SEQ ID NO: 26, SEQ ID NO: 27, SEQ ID NO: 28, SEQ ID NO: 29, SEQ ID NO: 34, SEQ ID NO: 35, SEQ ID NO: 38, SEQ ID NO: 39, SEQ ID NO: 40, SEQ ID NO: 41, SEQ ID NO: 43, and optionally SEQ ID NO: 46, SEQ ID NO: 47 and SEQ ID NO 48;
SEQ ID NO: 25, SEQ ID NO: 26, SEQ ID NO: 27, SEQ ID NO: 34, SEQ ID NO: 35, SEQ ID NO: 39, SEQ ID NO: 40, SEQ ID NO: 41, SEQ ID NO: 43;
SEQ ID NO: 25, SEQ ID NO: 26, SEQ ID NO: 27, SEQ ID NO: 28, SEQ ID NO: 34, SEQ ID NO: 35, SEQ ID NO: 39, SEQ ID NO: 40, SEQ ID NO: 43, and optionally SEQ ID NO: 46;
SEQ ID NO: 25, SEQ ID NO: 26, SEQ ID NO: 27, SEQ ID NO: 28, SEQ ID NO: 29, SEQ ID NO: 34, SEQ ID NO: 38, SEQ ID NO: 39, SEQ ID NO: 40, SEQ ID NO: 43;
SEQ ID NO: 25, SEQ ID NO: 26, SEQ ID NO: 27, SEQ ID NO: 34, SEQ ID NO: 35, SEQ ID NO: 38, SEQ ID NO: 39, SEQ ID NO: 40, SEQ ID NO: 41, SEQ ID NO: 43, and optionally SEQ ID NO: 46, SEQ ID NO: 47 and SEQ ID NO 48;
SEQ ID NO: 25, SEQ ID NO: 26, SEQ ID NO: 27, SEQ ID NO: 28, SEQ ID NO: 34, SEQ ID NO: 35, SEQ ID NO: 38, SEQ ID NO: 39, SEQ ID NO: 40, SEQ ID NO: 41, SEQ ID NO: 43;
SEQ ID NO: 25, SEQ ID NO: 26, SEQ ID NO: 27, SEQ ID NO: 28, SEQ ID NO: 34, SEQ ID NO: 35;
SEQ ID NO: 25, SEQ ID NO: 26, SEQ ID NO: 27, SEQ ID NO: 28, SEQ ID NO: 29, SEQ ID NO: 34, SEQ ID NO: 38;
SEQ ID NO: 25, SEQ ID NO: 26, SEQ ID NO: 27, SEQ ID NO: 34, SEQ ID NO: 35, SEQ ID NO: 38;
SEQ ID NO: 25, SEQ ID NO: 26, SEQ ID NO: 27, SEQ ID NO: 28, SEQ ID NO: 34, SEQ ID NO: 35, SEQ ID NO: 38;
SEQ ID NO: 25, SEQ ID NO: 26, SEQ ID NO: 27, SEQ ID NO: 28, SEQ ID NO: 29;
SEQ ID NO: 25, SEQ ID NO: 26, SEQ ID NO: 27, SEQ ID NO: 28; and
SEQ ID NO: 25, SEQ ID NO: 26, SEQ ID NO: 27

The amount of an lncRNA biomarker can also be determined from a biological sample by serial analysis of gene expression (SAGE), immunoassay, mass spectrometry, and any sequencing-based methods known in the art.

### Chip

The present invention also provide a Chip, a bioChip or a microarray, for the detection and/or quantification of the lncRNA biomarker(s) sequence(s) or fragment thereof according to the present invention, and a further study based on their expression profile for prostate cancer diagnosis purposes.

Such Chip may comprise: a solid support with organized immobilized probes, such probes being specifically complementary to at least one lncRNA biomarker as defined above or any combination thereof, in particular those specified above.

The probes on the Chip have addressable locations containing a characteristic associated therewith, that allows the detection and/or the quantification of multiple lncRNA biomarkers independently.

The solid support may be from a variety of materials commonly used in the field of gene chips, such as, but not limited to a nylon membrane, silicon-modified glass slides, glass unmodified sheet, plastic sheet or the like.

The Chip can be prepared in art-known conventional method for manufacturing a biochip. For example, if the solid support is used in a modified slide or silicon wafer, a probe 5′end a poly-dT sequence containing amino-modified oligonucleotide probes may be formulated as a solution, and then using a spotter which point in the modified slide or silicon wafer, are arranged in a predetermined sequence or array, and then fixed by overnight, can be obtained according to the present invention miRNA microarray.

Preferably, the Chip comprises probes allowing the detection of a lncRNA biomarker signature, such signature comprising or consisting of a combination of probes specifically complementary to lncRNA biomarkers as defined above or any combination thereof, in particular those specified above.

### Comparison to a Control Reference Value and Treatment

In an embodiment of the above mentioned methods, the method further comprises the step of comparing the amount of lncRNA biomarker in a biological sample to a reference or control amount. In particular, the reference amount can be the amount of the same lncRNA biomarker in a normal sample, i.e. a sample from a subject that did not have a prostate cancer. The normal sample may be obtained from the subject affected with the prostate cancer before the beginning of the disease or from another subject, preferably a normal or healthy subject, i.e. a subject who does not suffer from a cancer, especially a prostate cancer. The reference amount can be an average of the amounts obtained with different normal samples from different subjects, preferably subjects that do not have cancer.

The amounts of lncRNA biomarkers obtained with subjects may also be normalized by using the amounts obtained with other RNAs which are known to have stable expression.

The RT-PCR or the Nanostring method, or the Chip disclosed herein may further comprise primers sufficient for the detection of one or more housekeeping genes, preferably housekeeping genes selected from the group consisting of RPL11, GAPDH, NOL7, GPATCH3, ZNF2 and ZNF346. Preferably, such primers are selected from the group consisting of SEQ ID NO: 49, SEQ ID NO: 50, SEQ ID NO: 51, SEQ ID NO: 52, SEQ ID NO: 53 and SEQ ID NO: 54. Particularly, the RT-PCR or the Nanostring method, or the Chip disclosed herein comprises primers of SEQ ID NO: 49 for the detection of RPL11, SEQ ID NO: 50 for the detection of GAPDH, SEQ ID NO: 51 for the detection of NOL7, SEQ ID NO: 52 for the detection of GPATCH3, SEQ ID NO: 53 for the detection of ZNF2 and SEQ ID NO: 54 for the detection of ZNF346, respectively.

In one embodiment, the amount at least one lncRNA biomarker of fragment thereof determined in a biological sample in comparison to a reference amount allows the classification of the tumor status of a subject (i.e. Low-risk (LR), Intermediate-risk (IR) and High-risk (HR) of developing a prostate cancer). Particularly, the expression level (i.e. amount) of at least one lncRNA biomarker or fragment thereof in comparison to a reference value as defined herein allows the discrimination of high- and intermediate-risk prostate tumors versus low-risk and normal tissues for the diagnosis of prostate cancer in a subject.

In a further embodiment of the above mentioned methods, the method further comprises the step of determining whether the amount of an lncRNA biomarker is dysregulated, on the basis of the comparison between the amount in the biological sample and the reference amount, a higher amount of the lncRNA biomarker being indicative of a higher susceptibility to have or to develop a prostate cancer.

The amount of an lncRNA biomarker in a biological sample is considered as significantly different (i.e. dysregulated) compared to the reference amount, if, optionally after normalization, differences are in the order of 2-fold higher than the reference amount or more. Preferably, the amount of an lncRNA biomarker in a biological sample is considered as dysregulated if the amount is at least 2.5-fold higher, or 3, 3.5, 4, 4.5 or 5-fold higher than the reference amount.

Preferably, the at least one lncRNA biomarker or fragment thereof is over-expressed at least 6 times more in prostate tumor tissues comparing to normal prostate tissue.

In a preferred embodiment, the present invention provides a method for detecting prostate cancer in a subject from a sample comprising the steps of: (i) determining the amount of at least one lncRNA biomarker or a fragment thereof in a biological sample from the subject; (ii) comparing the amount in the sample to a reference amount derived from the amount of said lncRNA biomarker in samples obtained from subjects who haven't a prostate cancer; and (iii) identifying the subject as having a prostate cancer or as having an increased risk to develop a prostate cancer when the amount of said lncRNA biomarker in the sample is greater than the reference amount and/or identifying the subject as not having a prostate cancer or as not having an increased risk to develop a prostate cancer when the amount of said lncRNA biomarker in the sample is equal or less than the reference amount.

Optionally, the method may further comprise performing a biopsy of the prostate of the subject to confirm that the subject has a prostate cancer when the amount of said lncRNA biomarker in the sample is greater than the reference amount. Alternatively, the method may further comprise observing a biopsy of the prostate of the subject to confirm that the subject has a prostate cancer when the amount of said lncRNA biomarker in the sample is greater than the reference amount.

In yet another aspect, the present method further comprises a step of treating the patient diagnosed as having a prostate cancer.

The treatment of the patient diagnosed as having a prostate cancer may comprise the administration to the patient of an effective amount of a therapeutic agent and/or the prostate resection.

The therapeutic agent may be selected from the group consisting of radiotherapeutic agents, hormonal therapy agents, chemotherapy agents, immunotherapy agents and monoclonal antibody therapy agents, preferably hormonal therapy agents.

It is understood that the administered dose of the therapeutic agent may be adapted by those skilled in the art according to the patient, the pathology, the mode of administration, etc. The dosage and regimen depends in particular on the stage and severity of the prostate cancer, the weight and general state of health of the patient and the judgment of the prescribing physician.

In some embodiments, the treatment includes one or more of open prostatectomy, minimally invasive laparoscopic robotic surgery, intensity modulated radiation therapy (IMRT), proton therapy, brachytherapy, cryotherapy, molecular-targeted therapy, vaccine therapy and gene therapy, hormone therapy, active surveillance, or a combination thereof.

A biomarker, such as an lncRNA or fragment thereof that is differentially expressed or detected in a biological sample as described herein, may be a prognostic or a predictive marker. Prognostic and predictive biomarkers are distinguishable. A prognostic biomarker may be associated with a particular condition or disease such as prostate cancer, but is based on data that does not include a non-treatment or non-diseased control group. A predictive biomarker is associated with a particular condition or disease such as prostate cancer, as compared to a non-treated, non-diseased or other relevant control group (e.g., a different stage of cancer). By including such a control group, a prediction can be made about the prognosis of a patient that cannot be made using a prognostic biomarker.

In a particular embodiment, the present invention provides a method to assess the efficiency of a prostate cancer treatment in a subject having a prostate cancer comprising the steps of: (i) administering a prostate cancer treatment, i.e. an effective amount of a therapeutic agent, to the subject, (ii) determining the amount of at least one lncRNA biomarker or a fragment thereof in a biological sample from the subject, (iii) comparing the amount in the sample to a reference amount derived from the amount of said lncRNA biomarker in samples obtained from subjects that do not have a prostate cancer, and (iv) identifying the method as efficient or the subject as having recovered from prostate cancer when the amount of said lncRNA biomarker in the sample is equal or less than the reference amount.

Optionally or alternatively, the amount at least one lncRNA biomarker or fragment thereof determined in a biological sample obtained after administration of an effective amount of a therapeutic agent may further be compare to the amount of said lncRNA biomarker in a sample from the same patient obtained before the treatment, a significant decrease in the amount of said lncRNA biomarker being indicative of the efficiency of said prostate cancer treatment.

### Subject

As used herein, the terms “subject”, “individual” or “patient” are interchangeable and refer to an animal, preferably to a mammal, even more preferably to a human. However, the term “subject” can also refer to non-human animals, in particular mammals such as dogs, cats, horses, cows, pigs, sheep and non-human primates, among others, that are in need of diagnostic.

In a preferred embodiment of the above mentioned method, the subject is a man, preferably an adult man, preferably a man of at least 45 years old, more preferably a man of at least 50 years old, even more preferably a man of at least 60 years old.

In a particular embodiment of the above mentioned method, the subject has a family history of prostate cancer or other risk factors. In this case, the subject can be a man of at least 40 years old.

In another particular embodiment of the above mentioned method, the subject has relapse from a previous prostate cancer.

In a preferred embodiment of the above mentioned method, the subject is positive to a digital-rectal examination and/or to a prostate specific antigen (PSA) test.

In a preferred embodiment of the above mentioned method, the subject is periodically submitted to a prostate cancer diagnosis, preferably once a year, alternatively every two years or every three years.

In a particular embodiment of the above mentioned method, when the subject has a family history of prostate cancer or other risk factors, the subject is submitted to a prostate cancer diagnosis twice a year.

### Kit and Use of a Kit

The invention also concerns a kit for the diagnosis of prostate cancer in a subject, wherein the kit comprises probes and/or primers capable to specifically hybridize to at least one lncRNA biomarker selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9, SEQ ID NO: 10, SEQ ID NO: 11, SEQ ID NO: 12, SEQ ID NO: 13, SEQ ID NO: 14, SEQ ID NO:20 and SEQ ID NO: 21, preferably, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9, SEQ ID NO:20 and SEQ ID NO: 21. Optionally, the kit may further comprise probes and/or primers for the detection of additional prostate cancer biomarkers, preferably probes and/or primers for the detection of lncRNAs prostate cancer biomarkers, more preferably probes and/or primers capable to specifically hybridize to at least one biomarker selected from the group consisting of SEQ ID NO: 15, SEQ ID NO: 16, SEQ ID NO: 17, SEQ ID NO: 18, SEQ ID NO: 19, SEQ ID NO: 22, SEQ ID NO: 23 and SEQ ID NO: 24.

Preferably, the kit comprises probes and/or primers capable to specifically hybridize to at least 2, at least 3, at least 4 at least 5, at least 6, at least 7 at least 8, at least 9, at least 10, at least 11, at least 12, at least 13, at least 14, at least 15, at least 16, at least 17, at least 18, or at least 19 lncRNA biomarkers as described above.

For instance, the kit may comprise probes and/or primers capable to specifically hybridize to a combination of biomarkers as defined above.

Optionally, the above mentioned kit further comprises a leaflet providing guidelines to use such a kit.

Probes and/or primers of the kit, as used herein, are as defined above in any embodiment.

Preferably, for each lncRNA biomarker, the kit comprises two probes, preferably Nanostring probes consisting of:
A capture-probe which comprises a nucleotide sequence hybridizing a first part of the lncRNA biomarker and a molecule able to bind a solid support; and
A reporter-probe which comprises a nucleotide sequence hybridizing a second part of the lncRNA biomarker and a detectable label.

Alternatively, for each lncRNA biomarker, the kit comprises two probes, preferably Nanostring probes consisting of:
A capture-probe which hybridizes the lncRNA biomarker and links a molecule that allows to immobilize the lncRNA biomarker onto a solid support; and
A reporter-probe which hybridizes the lncRNA biomarker and links a label that allows the detection of the immobilized lncRNA biomarker.

Preferably, the immobilizing agent comprises biotin.

Preferably, the label is a fluorescent label, more preferably a combination of several fluorophores, even more preferably a combination of six fluorophores selected from four different fluorophores.

Preferably, the kit comprises at least two pairs of probes selected from the pairs of probes above mentioned. More preferably, the kit comprises at least the three pairs of probes above mentioned.

When several pairs of probes are included in the kit, the label is different for each of the probes presenting a label.

Alternatively, for each lncRNA biomarker, the kit comprises:
at least one primer pair;
optionally, RT primers, preferably random primers, alternatively a RT primer complementary to said at least one lncRNA biomarker.
optionally, a labeled probe.

Preferably, the labeled probe, is a fluorescent probe, more preferably a dual labeled probe which is complementary to a sequence of the target sequence localized between the two primers, even more preferably, the dual label probe is aimed to fluoresce upon 5′→3′ exonuclease activity.

Preferably, the at least one primer pair consists in primers that are complementary to exon sequence of the lncRNA, and are obligatory out of exon of the sense-paired pre-mRNA, if said lncRNA is an antisense lncRNA. More preferably, the primers are complementary to two adjacent exon sequences of the lncRNA (positioned upstream and downstream of the exon-exon junction), and are obligatory out of exons of the sense-paired pre-mRNA if said lncRNA is an antisense lncRNA.

The above mentioned combination may further comprise additional primer pairs, preferably primer pairs targeting other lncRNA which are diagnosis marker for prostate cancer, more preferably primer pairs for PCA3 quantitative amplification.

Alternatively, the kit may comprises a Chip or microarray as described hereabove.

The invention also concerns the use of a kit as described above in the diagnosis of prostate cancer in a subject.

Preferably, the subject is an animal, more preferably a mammal, even more preferably a human.

In a most preferred embodiment, the subject is an adult man of at least 50 years old.

Further aspects and advantages of the present invention will be described in the following examples, which should be regarded as illustrative and not limiting.

The invention also relates to a composition or set of nucleic acid primers comprising a plurality of nucleic acid primers, said plurality comprising primers that specifically amplify each lncRNA of a lncRNA profile or signature characteristic of prostate cancer as disclosed herein. In this regard, the invention particularly relates to a set of nucleic acid primers comprising a plurality of nucleic acid primers, said plurality comprising primers that specifically amplify distinct lncRNAs selected from lncRNAs of SEQ ID NO: 1-24. Such primers preferably comprises SEQ ID NOs: 25-54, respectively.

### Examples

### Results

Validation of Contigs Expression in Prostate Tumors by the NanoString nCounter Assay

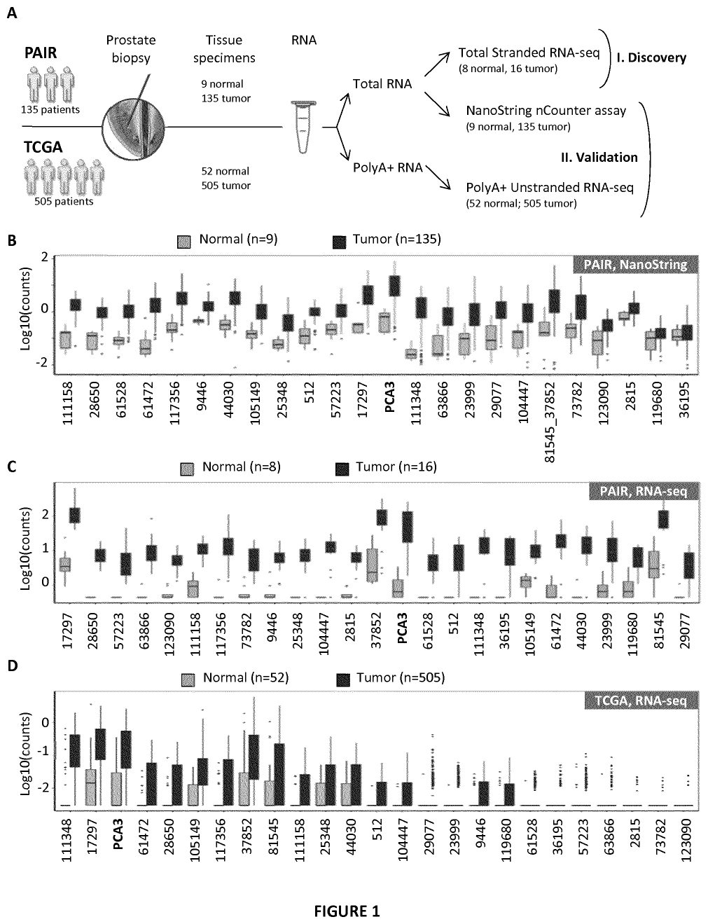

The inventors identified contigs as PCa biomarkers. A contig is a set of overlapping nucleic acid segments that together represent a consensus sequence. More specifically, the inventors queried their expression in an extended PAIR cohort and in TCGA-PRAD (FIG. 1a). First, expression of contigs was measured in the enlarged PAIR cohort of 9 normal and 135 tumor specimens using the NanoString nCounter™ platform for direct enzyme-free multiplex digital RNA counting (Table 1).

TABLE 1
Clinico-pathological characteristics, risk classification
and recurrence status of the prostate specimens used
in the NanoString assay (PAIR, NanoString dataset).

| | Number of specimens |
|---|---|
| Normal | 9 |
| Tumor | 135 |
| High risk | 49 |
| Intermediate risk | 51 |
| Low risk | 35 |
| No relapse | 79 |
| Relapse | 55 |
| Gleason score | |
| 6 | 22 |
| 7 | 79 |
| 8 | 29 |
| 9 | 6 |
| TNM | |
| PT2a | 7 |
| pT2b | 1 |
| pT2c | 37 |
| pT3a | 61 |
| pT3b | 22 |
| pT4 | 7 |

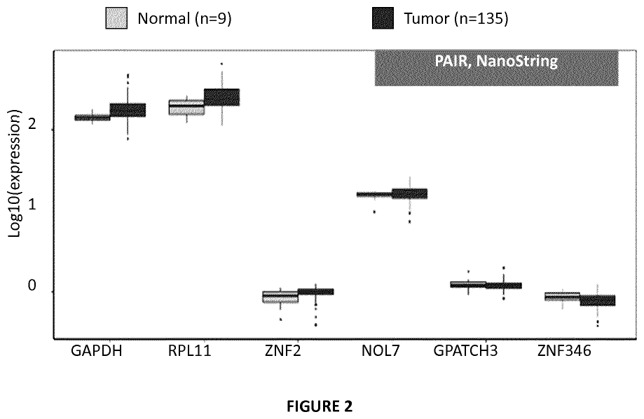

In addition to the selected 23 contigs, a probe for PCA3 was used as a benchmark for the prostate cancer lncRNA. The inventors also monitored the expression of 6 housekeeping genes (RPL11, GAPDH, NOL7, GPATCH3, ZNF2, ZNF346) and selected 3 lowly expressed mRNAs (GPATCH3, ZNF2, ZNF346) as custom internal controls for relative quantifications (FIG. 2). This assay revealed that all contigs were expressed to lower level than PCA3, but still 21 out of 23 contigs were significantly overexpressed (mean fold-change, FC>2 and Wilcoxon p-value<0.05) in tumor specimens (FIG. 1b). Two contigs, intergenic ctg_119680 and repeat ctg_36195 showed no differential expression between normal and tumor tissues. Ranking according to DE performance (p-values) showed 12 contigs better than PCA3 (namely, 111158, 28650, 61528, 61472, 117356, 9446, 44030, 105149, 25348, 512, 57223, 17297). Among the top DE contigs were those embedded into PCAT1 (ctg_105149), CTBP1-AS (ctg_25348) and PCAT7 (ctg_111158) genes, but the rest were assigned to novel lncRNAs. Apart from ctg_36195 and ctg_119680, expression measurements were generally consistent across technologies (total stranded RNA-seq vs. NanoString), though the DE-performance ordering was different (FIG. 1c). This allowed validation of at least 21 contigs candidates for further analysis.

### Validation of Contigs Expression in the TCGA RNA-Seq Dataset

The inventors measured the occurrence of sequences representing the 23 contigs and the PCA3 probe across the TCGA prostate cancer polyA+RNA-seq libraries (TCGA-PRAD cohort), including 52 normal and 507 tumor specimens. The TCGA RNA-seq dataset differs remarkably from ours in terms of library preparation and sequencing protocols. In particular, selection of polyadenylated RNA species leads to depletion of lncRNAs with poorly or non-polyadenylated 3′ RNA ends, as well as to a poorer coverage of 5′ RNA ends. Moreover, the unstranded TCGA libraries may compromise both discrimination and counting of sense/antisense paired transcripts.

In spite of all these limitations, at least 16 out of 23 contigs had significant support (p-value<0.01, FC>2) for overexpression in tumor specimens in the TCGA dataset (FIG. 1d). Among the best scored candidates were two novel contigs, one antisense to DLX1 (ctg_111348) and another intergenic (ctg_17297), both overperforming PCA3 ranked third. On the other hand, at least 9 contigs were near silent in the TCGA dataset. This was independent of their genomic (intergenic or antisense) location and of expression of paired sense gene (data not shown). Detection of these contigs in TCGA data may thus be compromised due to relatively low RNA-seq coverage or to transcripts depletion because of their poor polyadenylation.

### Specificity of Contigs Expression in Other Tissues

In a perspective of development of the urinary diagnostic tests the inventors evaluated the specificity of the contigs expression in other tissues, particularly, in bladder and kidney that could be harvested together with prostatic cells from urine following prostate massage. First, in parallel with prostate specimens the inventors measured the expression of contigs in bladder tissues (2 normal and 8 tumor specimens) by the NanoString assay (Table 2). Remarkably, the abundance of all PCA3 and contigs was extremely low in these specimens.

TABLE 2
Clinico-pathological characteristics of bladder specimens.

| Sample ID | Stage | Grade |
|---|---|---|
| IC_BLCA_5 | T4 | / |
| IC_BLCA_7 | T4a | G3 |
| IC_BLCA_9 | T4a | G3 |
| IC_BLCA_11 | T3 | G3 |
| IC_BLCA_14 | T3a | G3 |
| IC_BLCA_19 | T3b | G3 |
| IC_BLCA_53 | Ta | G2 |
| IC_BLCA_60 | T1a | G2 |
| IC_BLCA_359 | Normal | / |
| IC_BLCA_361 | Normal | / |

### Retrieval of Contigs for Detection of PCa Independent of Tumor Risk and Recurrence Status

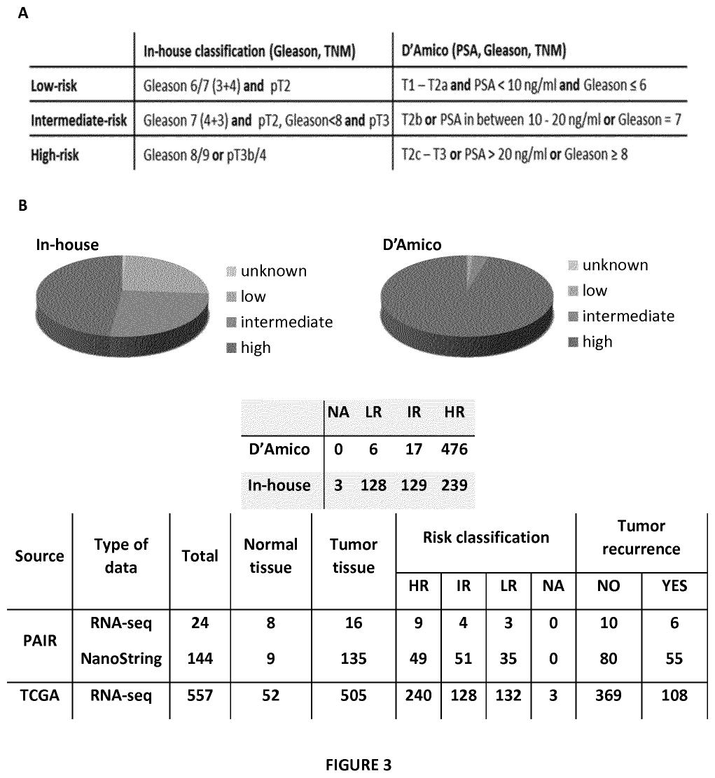

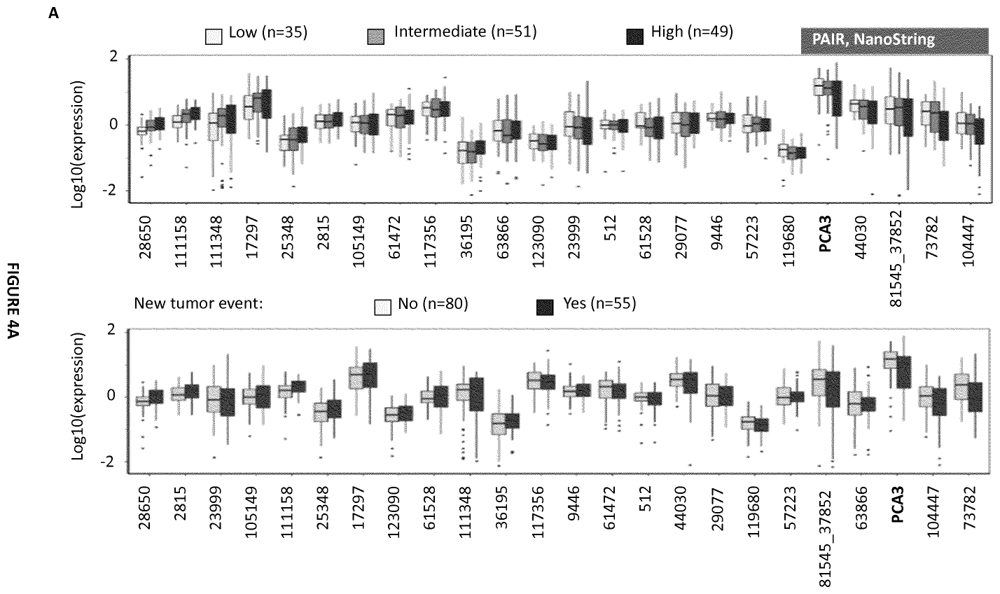

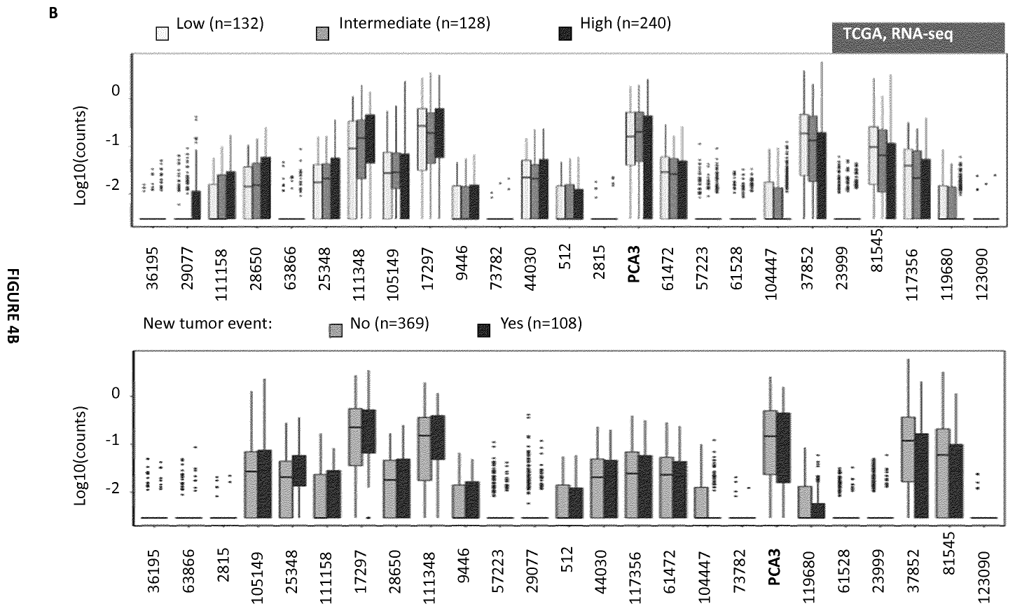

With the ultimate goal to conceive a molecular diagnostic tool independent of the actual tumor staging, risk prognosis and recurrence status, the inventors compared expression of contigs in tumors of different clinical characteristics. For risk prognosis, the most commonly used is the three-group risk stratification system established by D'Amico in 1998 which takes into account preoperative PSA level, biopsy Gleason score and clinical TNM stage. As mentioned above, this scheme is highly debated because of the high controversial PSA score. To define a molecular signature independent of the PSA score, the inventors eliminated this criterion and categorized tumor specimens into low-, intermediate- and high-risk groups uniquely on the basis of Gleason and TNM features (FIG. 3a). Exclusion of the PSA score led to a decrease in high-risk and an increase in low- and intermediate-risk tumors in the TCGA-PRAD cohort compared to D'Amico based grouping (FIG. 3b). In addition to risk assessment, the inventors also separated TCGA and PAIR cohorts' specimens in two groups depending on the tumor recurrence status (FIG. 3c). Then, expression of PCA3 and contigs were compared between these groups (FIG. 4). As previously reported, PCA3 expression was more disperse with lower mean expression in high-risk tumors and in patients presenting a new tumor event than the majority of contigs in both PAIR and TCGA-PRAD cohorts. In contrast, the majority of contigs showed consistent and high expression independently of tumor risk and recurrence status.

While ranking by decreasing FC of mean expression, 17 out of 21 and 9 out of 15 contigs outperformed PCA3 in the PAIR and TCGA cohorts, respectively, among them intergenic ctg_17297 was among the best. Robust expression independent of tumor risk and recurrence status additionally support the value of these contigs as putative PCa biomarkers.

### Diagnostic Performance of Contigs in Different Datasets

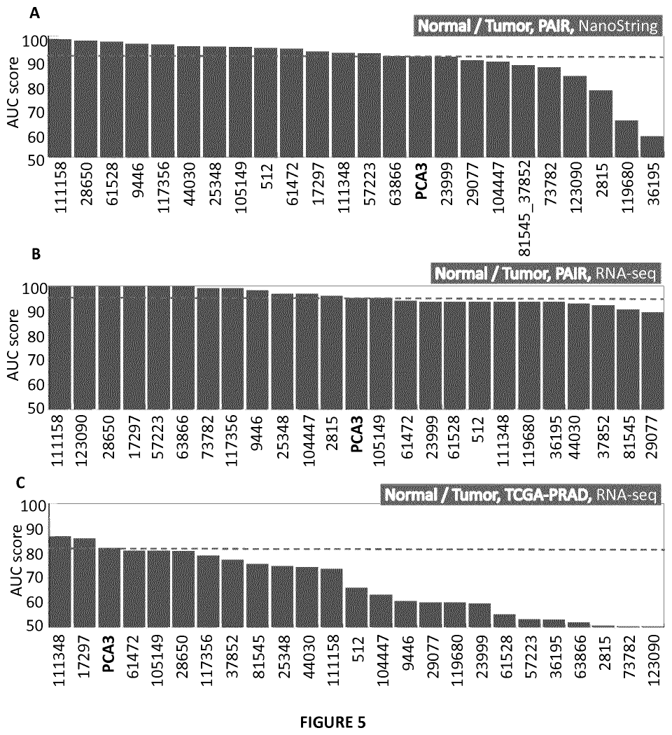

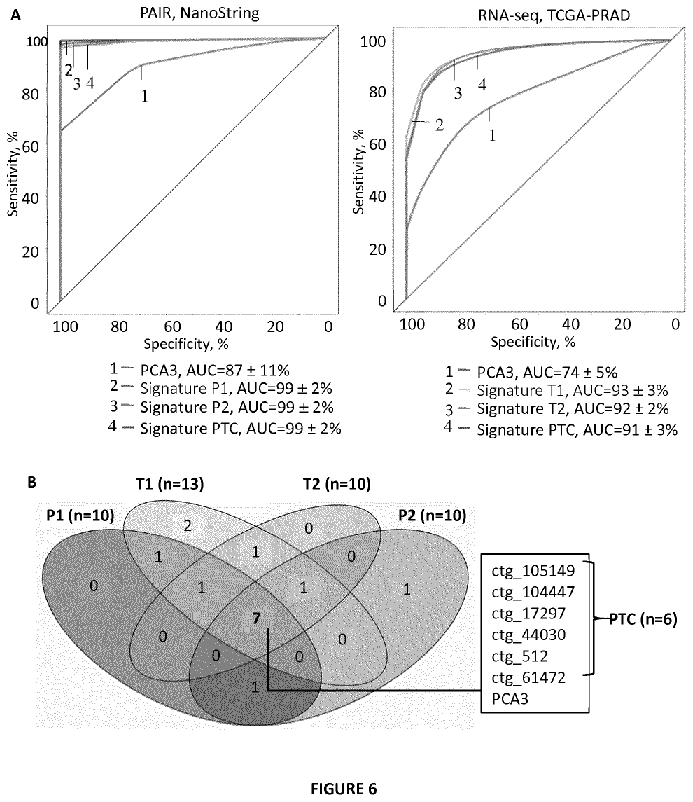

To assess the sensitivity of contigs in PCA diagnosis the inventors performed receiver operating characteristic (ROC) curve analysis across PAIR and TCGA-PRAD cohorts for NanoString and RNA-seq expression counts. AUC scores were well correlated with DE-scores (FIG. 5). Expectedly, performance of contigs in the PAIR datasets were self-consistent and characterized by much higher AUC values than in TCGA-PRAD (FIG. 6).

In total, 12 contigs overcame PCA3 in DE-score and diagnostic performance in the PAIR cohort. Among them, contigs antisense to DLX1 (ctg_111348) and intergenic ctg_17297 also outperformed PCA3 in TCGA-PRAD (FIG. 5c).

### Inferring High Performance Multiplex Signatures for PCa Diagnostics

The inventors applied logistic lasso regression to extract parsimonious probe subsets predicting PCa independently of the tumor status. Starting from the complete set of probes including or excluding PCA3, the procedure applied to the PAIR and TCGA-PRAD datasets selected 10 and 13 candidates, respectively (Signatures P1 and T1, Table 3). Both signatures contained PCA3, contigs within PCAT7 and PCAT1 but also contigs within novel lncRNAs. Selected probes were then used to construct a tumor predictor using K-fold cross-validated logistic regression. Both multiplex signatures markedly outperformed PCA3 for tumor detection (AUC=0.99 vs. 0.87 in PAIR and AUC=0.93 vs. 0.74 in TCGA-PRAD, respectively) (FIG. 6a, Table 3) either when the signature includes or excludes PCA3.

Notably, this signature was also better at predicting high-risk tumors. Discrimination of high- and intermediate-risk tumors versus low-risk and normal tissues was also improved, albeit at a lower performance level (Table 3).

In order to design a PCa-specific diagnostic tool, the same variable selection and predictor construction was employed but on the subset of probes expressed only in prostate tumors and not in other tissues. This excluded contigs assigned to PCAT7 (ctg_111158, ctg_28650), CTBP1-AS (ctg_25348), ctg_9446 and ctg_63866 were excluded. The resulting signatures P2 and T2 achieved nearly equal performances for tumor prediction independently of its status as signatures built from all probes, the signature including or excluding PCA3 biomarker (Table 3). Intersection of the PAIR and TCGA-PRAD signatures included 7 candidates (FIG. 6b). A manually selected set of 23 contigs was further validated using different clinical and experimental setups and tested for diagnostic performance by ROC and logic regression analysis. This defined a restricted contig panel as potent diagnostic biomarkers of PCa independent of patho-clinical characteristics overperforming the currently used PCA3.

TABLE 3
Prediction performance (mean and standard deviation of area under ROC curve, AUC) of PCA3
alone and contig based signatures in the PAIR NanoString and TCGA-PRAD polyA+ RNA-
seq datasets: P1 and T1 were inferred from complete set of probes, P2 and T2 from prostate
specific subset. PTC signature is composed of common PAIR and TCGA contigs excluding PCA3.

| | NanoString | | | | | |
|---|---|---|---|---|---|---|
| | PCA3 | P1 | P1 − PCA3 | P2 | P2 − PCA3 | PTC |
| Normal vs Tumor | 0.88 ± 0.09 | 1.00 ± 0.01 | 0.99 ± 0.01 | 0.99 ± 0.01 | 0.99 ± 0.02 | 0.99 ± 0.02 |
| Normal vs HR | 0.82 ± 0.12 | 0.98 ± 0.06 | 0.99 ± 0.02 | 0.99 ± 0.03 | 0.98 ± 0.03 | 0.99 ± 0.02 |
| Normal vs IR | 0.89 ± 0.10 | 0.98 ± 0.06 | 0.96 ± 0.09 | 0.98 ± 0.05 | 0.97 ± 0.06 | 0.97 ± 0.08 |
| Normal vs LR | 0.94 ± 0.10 | 0.95 ± 0.08 | 0.960 ± 0.07 | 0.94 ± 0.10 | 0.93 ± 0.13 | 0.96 ± 0.10 |
| LR vs HR | 0.66 ± 0.07 | 0.74 ± 0.09 | 0.76 ± 0.08 | 0.71 ± 0.09 | 0.66 ± 0.08 | 0.68 ± 0.09 |

TCGA-PRAD RNA-seq

| | PCA3 | T1 | T1 − PCA3 | T2 | T2 − PCA3 | PTC |
|---|---|---|---|---|---|---|
| Normal vs Tumor | 0.74 ± 0.04 | 0.93 ± 0.03 | 0.93 ± 0.03 | 0.92 ± 0.03 | 0.92 ± 0.04 | 0.91 ± 0.03 |
| Normal vs HR | 0.69 ± 0.05 | 0.92 ± 0.04 | 0.93 ± 0.03 | 0.90 ± 0.04 | 0.90 ± 0.04 | 0.89 ± 0.03 |
| Normal vs IR | 0.78 ± 0.05 | 0.94 ± 0.03 | 0.93 ± 0.03 | 0.94 ± 0.03 | 0.92 ± 0.04 | 0.91 ± 0.04 |
| Normal vs LR | 0.78 ± 0.05 | 0.91 ± 0.03 | 0.92 ± 0.03 | 0.92 ± 0.04 | 0.92 ± 0.04 | 0.92 ± 0.04 |
| LR vs HR | 0.59 ± 0.03 | 0.64 ± 0.05 | 0.63 ± 0.05 | 0.59 ± 0.04 | 0.60 ± 0.05 | 0.55 ± 0.04 |

Signatures:

P1 (n=10): ctg_73782, ctg_104447, PCA3, ctg_105149, ctg_117356, ctg_17297, ctg_28650, ctg_44030, ctg_512, ctg_61472.

P1-PCA3: ctg_73782, ctg_104447, ctg_105149, ctg_117356, ctg_17297, ctg_28650, ctg_44030, ctg_512, ctg_61472.

P2 (n=10): ctg_73782, ctg_104447, PCA3, ctg_105149, ctg_111348, ctg_17297, ctg 2815 ctg_44030, ctg_512, ctg_61472.

P2-PCA3: ctg_73782, ctg_104447, ctg_105149, ctg_111348, ctg_17297, ctg_2815, ctg_44030, ctg_512, ctg_61472.

T1 (n=13): PCA3, ctg_105149, ctg_111158, ctg_25348, ctg_104447, ctg_23999, ctg_61472, ctg_28650, ctg_117356, ctg_44030, ctg_512, ctg_17297, ctg_111348.

T1-PCA3: ctg_105149, ctg_111158, ctg_25348, ctg_104447, ctg_23999, ctg_61472, ctg_28650, ctg_117356, ctg_44030, ctg_512, ctg_17297, ctg_111348.

T2 (n=10): PCA3, ctg_105149, ctg_104447, ctg_23999, ctg_61472, ctg_117356, ctg_44030, ctg_512, ctg_17297, ctg_111348.

T2-PCA3: ctg_105149, ctg_104447, ctg_23999, ctg_61472, ctg_117356, ctg_44030, ctg_512, ctg_17297, ctg_111348.

PTC (n=6): ctg_105149, ctg_104447, ctg_17297, ctg_44030, ctg_512, ctg_61472.

TABLE 4
Correspondence between SEQ ID NOs
and contig references (ctg_number).

| SEQ ID NO: | Contig reference |
|---|---|
| 1 | ctg_44030 |
| 2 | ctg_512 |
| 3 | ctg_104447 |
| 4 | ctg_73782 |
| 5 | ctg_2815 |
| 6 | ctg_61528 |
| 7 | ctg_119680 |
| 8 | ctg_36195 |
| 9 | ctg_123090 |
| 10 | ctg_17297 |
| 11 | ctg_117356 |
| 12 | ctg_29077 |
| 13 | ctg_57223 |
| 14 | ctg_111348 |
| 15 | ctg_105149 |
| 16 | ctg_61472 |
| 17 | ctg_23999 |
| 18 | ctg_81545_37852 |
| 19 | PCA3 |
| 20 | ctg_9446 |
| 21 | ctg_63866 |
| 22 | ctg_28650 |
| 23 | ctg_111158 |
| 24 | ctg_25348 |

### Material and Methods

### Tissue Samples

Tumor and normal biopsy specimens were collected from prostate cancer patients who provided informed consent and were approved for distribution by the H. Mondor institutional board (PAIR cohort). Tumors classification in low-, intermediate- and high-risk prognosis was performed according to Gleason and TNM scores and regardless PSA values.

RNA Extraction, Quantification and cDNA Library Production

Total RNA was extracted using the TRizol reagent (ThermoFisher), according to manufacturer's procedure, quantified and quality controlled using a 2100 Bioanalyzer (Agilent). RNA samples with RNA Integrity Number (RIN) above 6 were depleted for ribosomal RNA and converted into cDNA library using a TruSeq Stranded Total Library Preparation kit (Illumina). cDNA libraries were normalized using an Illumina Duplex-specific Nuclease (DSN) protocol prior to a paired-end sequencing on HiSeq™ 2500 (Illumina). At least 20× coverage per sample was considered as minimum of unique sequences for further data analysis.

### RNA-Sequencing Datasets

Raw paired-end strand-specific RNA-seq data was generated from ribo-depleted total RNA samples of prostate tissues (8 normal and 16 tumor specimens; Supplementary Table 1) and can be retrieved from the gene omnibus portal (GEO), accession number GSE115414. TCGA prostate cancer polyA-selected RNA-seq and corresponding clinical data were obtained from publicly available TCGA dataset (http://cancergenome.nih.gov), 557 inputs in total (52 normal and 505 tumors of high- (n=240), intermediate- (n=128) and low-risk (n=132) groups. Among them, 369 patients showed no tumor recurrence, 108 presented a new tumor event (FIG. 3c).

### Signature of Prostate Tumor Tissues

Read counting was performed on the compiled annotation (GENCODE v26, HoLdUp Class1 and Class2) for each sample, using featureCounts 1.6.0 with the following parameters: -F “SAF”-p -s 2 -O and the DESeq R package. Only RNAs with adjusted p-value below 0.01 were retained as differentially expressed to constitute the prostate tumor signature.

NanoString nCounter Expression Assay

100 ng of total RNA was used for direct digital detection of 30 target transcripts: 6 housekeeping genes (RPL11, GAPDH, NOL7, GPATCH3, ZNF2 and ZNF346), 23 contigs and the one known PCa-associated lncRNA, PCA3. Each target gene of interest was detected in RNA samples of 144 prostate specimens (9 normal and 135 tumor) of the PAIR cohort on NanoString nCounter V2 using reporter and capture probes of 35- to 50-nts targeting sequences listed in Table 10. Data was normalized through the use of NanoString's intrinsic positive controls and then contig expression was calculated relative to the average of signal of lowly expressed housekeeping genes (GPATCH3, ZNF2 and ZNF346).

### Receiver Operating Curve (ROC) Analysis

ROC curves assessed the assignment of 135 tumor and 9 normal prostate specimens for each probe (PCA3 and contigs), using pROC R-package and log 2(expression) from NanoString and RNA-seq data of PAIR and TCGA cohorts.

### RNA-Sequencing Data Visualization

RNA-seq reads profiling along a locus of interest was performed using an in-house R script VING. The normal samples were assigned to the group “controls”, and the tumor samples—to the group “cases”, with the assumption the “cases” should have higher values than the “controls”.

### Unsupervised Clustering of Prostate Specimens

Specimens were ranked based on the Log 10(expression) levels of contigs assessed by NanoString nCounter assay using ComplexHeatmap R-package 30.

### Variable Selection and Model Building

Signature inference was performed in R, applying the same procedure to either the PAIR NanoString expression table (24 probes×144 observations) or the TCGA-PRAD contig count table (25 probes×557 observations). First, the inventors performed penalized logistic regression using the glmnet R package to select probes predicting the tumor status. Selection was performed on all probes including PCA3 (signatures P1 & T1), prostate-specific probes including PCA3 (signatures P2 & T2). Second, the inventors built predictors using boosted logistic regression from the caTools and caret packages. To assess prediction performance, AUCs were computed using the precrec package on 100 training and testing datasets, subsampled from the initial dataset (Normal vs. Tumor) using the sample.split function from the caTools package. The inventors assessed prediction performance on subsets of the initial datasets (Normal vs. High Risk, Normal vs. Low Risk, etc) using the same procedure. The R code and data tables are provided in supplementary files.

1-16. (canceled)

17. An in vitro method for prostate cancer diagnosis of a subject, wherein the method comprises the step of determining the amount of at least one biomarker in a biological sample from said subject, said at least one biomarker being selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20, SEQ ID NO: 21, SEQ ID NO: 22 and any fragment thereof of at least 30 nucleotides, and wherein an increased amount of the at least one biomarker is indicative of prostate cancer.

18. The method according to claim 17, wherein the at least one biomarker is selected the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 20, SEQ ID NO: 21 and any fragment thereof of at least 30 nucleotides.

19. The method according to claim 17, wherein the at least one biomarker is used in combination with at least one additional biomarker selecting from the group consisting of SEQ ID NO: 15, SEQ ID NO: 16, SEQ ID NO: 17, SEQ ID NO: 18, SEQ ID NO: 19, SEQ ID NO: 23, SEQ ID NO: 24 and any fragment thereof of at least 30 nucleotides.

20. The method according to claim 17, wherein at least three, four, five, six, seven or eight biomarkers are used in combination and wherein said combination are selected in any one of the following groups consisting of:
SEQ ID NO: 1, SEQ ID NO: 2, SEQ ID NO: 3, SEQ ID NO: 4, SEQ ID NO: 10, SEQ ID NO: 11 and a fragment of at least 30 nucleotides thereof;
SEQ ID NO: 1, SEQ ID NO: 2, SEQ ID NO: 3, SEQ ID NO: 4, SEQ ID NO: 5, SEQ ID NO: 10, SEQ ID NO: 14 and a fragment of at least 30 nucleotides thereof;
SEQ ID NO: 1, SEQ ID NO: 2, SEQ ID NO: 3, SEQ ID NO: 10, SEQ ID NO: 11, SEQ ID NO: 14, and a fragment of at least 30 nucleotides thereof; and
SEQ ID NO: 1, SEQ ID NO: 2, SEQ ID NO: 3, SEQ ID NO: 4, SEQ ID NO: 5, SEQ ID NO: 10, SEQ ID NO: 11, SEQ ID NO: 14 and a fragment of at least 30 nucleotides thereof.

21. The method according to claim 17, wherein the combination comprises or consists of any one of the following combinations of biomarkers:
SEQ ID NO: 1, SEQ ID NO: 2, SEQ ID NO: 3, SEQ ID NO: 4, SEQ ID NO: 10, SEQ ID NO: 11 and a fragment of at least 30 nucleotides thereof;
SEQ ID NO: 1, SEQ ID NO: 2, SEQ ID NO: 3, SEQ ID NO: 4, SEQ ID NO: 5, SEQ ID NO: 10, SEQ ID NO: 14 and a fragment of at least 30 nucleotides thereof;
SEQ ID NO: 1, SEQ ID NO: 2, SEQ ID NO: 3, SEQ ID NO: 10, SEQ ID NO: 14, and a fragment of at least 30 nucleotides thereof;
SEQ ID NO: 1, SEQ ID NO: 2, SEQ ID NO: 3, SEQ ID NO: 10 and a fragment of at least 30 nucleotides thereof; or
SEQ ID NO: 1, SEQ ID NO: 2, SEQ ID NO: 3, SEQ ID NO: 10, SEQ ID NO: 11, SEQ ID NO: 14 and a fragment of at least 30 nucleotides thereof.

22. The method according to claim 21, wherein the combination further comprises one, two, three or four biomarkers selected in the group consisting of SEQ ID NO: 15, SEQ ID NO: 16, SEQ ID NO:17, SEQ ID NO: 19 and any fragment of at least 30 nucleotides thereof.

23. The method according to claim 22, wherein the combination comprises or consists of the following combinations of biomarkers:
SEQ ID NO: 1, SEQ ID NO: 2, SEQ ID NO: 3, SEQ ID NO: 4, SEQ ID NO: 10, SEQ ID NO: 11, SEQ ID NO: 15, SEQ ID NO: 16, SEQ ID NO: 19 and a fragment of at least 30 nucleotides thereof, and optionally SEQ ID NO: 22 or a fragment of at least 30 nucleotides thereof;
SEQ ID NO: 1, SEQ ID NO: 2, SEQ ID NO: 3, SEQ ID NO: 4, SEQ ID NO: 5, SEQ ID NO: 10, SEQ ID NO: 14, SEQ ID NO: 15, SEQ ID NO: 16, SEQ ID NO: 17, SEQ ID NO: 19 and a fragment of at least 30 nucleotides thereof;
SEQ ID NO: 1, SEQ ID NO: 2, SEQ ID NO: 3, SEQ ID NO: 10, SEQ ID NO: 11, SEQ ID NO: 14, SEQ ID NO: 15, SEQ ID NO: 16, SEQ ID NO: 17, SEQ ID NO: 19 and a fragment of at least 30 nucleotides thereof, and optionally SEQ ID NO: 22 and/or SEQ ID NO: 23 and/or SEQ ID NO: 24 or a fragment of at least 30 nucleotides thereof;
SEQ ID NO: 1, SEQ ID NO: 2, SEQ ID NO: 3, SEQ ID NO: 10, SEQ ID NO: 11, SEQ ID NO: 14, SEQ ID NO: 15, SEQ ID NO: 16, SEQ ID NO: 17, SEQ ID NO: 19 and a fragment of at least 30 nucleotides thereof;
SEQ ID NO: 1, SEQ ID NO: 2, SEQ ID NO: 3, SEQ ID NO: 10, SEQ ID NO: 14, SEQ ID NO: 15, SEQ ID NO: 16, SEQ ID NO: 19 and a fragment of at least 30 nucleotides thereof; or
SEQ ID NO: 1, SEQ ID NO: 2, SEQ ID NO: 3, SEQ ID NO: 10, SEQ ID NO: 15, and SEQ ID NO: 16 and a fragment of at least 30 nucleotides thereof.

24. The method according to claim 17, said method discriminating high- and intermediate-risk of developing a prostate cancer versus low-risk of developing a prostate cancer in a subject.

25. The method according to claim 17, wherein said sample is a body fluid, blood sample or urine sample.

26. The method according to claim 17, wherein the biomarker amount is determined by amplification or by hybridization.

27. The method according to claim 17, wherein the subject is a mammal, a human, or a man of at least 40 years old.

28. A kit for the diagnosis of prostate cancer in a subject, wherein the kit comprises (i) probes and/or primers capable to specifically hybridize to at least one biomarker selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20 and SEQ ID NO: 21; and optionally, a leaflet providing guidelines to use said kit.

29. The kit according to claim 28, wherein the kit further comprises probes and/or primers for the detection of additional prostate cancer biomarkers and/or primers capable to specifically hybridize to at least one biomarker selected from the group consisting of SEQ ID NO: 15, SEQ ID NO: 16, SEQ ID NO: 17, SEQ ID NO: 18, SEQ ID NO: 19, SEQ ID NO: 22, SEQ ID NO: 23 and SEQ ID NO: 24.

30. An isolated nucleic acid comprising or consisting of a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 20 and SEQ ID NO: 21 and any fragment thereof of at least 30 nucleotides or a complementary sequence thereof, wherein the nucleic acid sequence has a length shorter than 3500 nucleotides.


SEQUENCE LISTING (54 sequences)

SEQ ID NO: 1 - RNA, 672 nt, Artificial Sequence
  ctg_44030
gggaacuacaauucaagauuuggguggggacacagccaaaccguaucagagcugugcucccaagcacccaaggacugccgaguucucaggcuggcagcuccagcugauggcacgucacuggggggugagggguugaggaccugagcugcccccaggcucugcaauggaaagcaucaggcuggacuuggacauucugggugggaagugucuggaaccuggacgcuggcuuggagaggagccucaggaggaaucgccaggauucucagcugucgagaaugucacccgcugcuguacucccaacagcaagggaggcaccuggcacgucgugagcaacucucguguauuuguugcaugaucuaguccaagaauggccaaugaguuucaccugccaguuuucacaggccugugugccgagaguguuccuuaccauuuuuucauuauuauucugcuaaggaggauuuuuagacauuauguuccuagucaagcccucaccucugcaaugaaauuauaauaccacuguugacccaaagaugagcagcaagcgcuagaacugagggaaaucauggugagauuuauucugagccaagcuggaggacuuaggcccaggaacacagaaucuguguccacugagaauguaucucaaagcaggcuccaugaggcaccgugaacuua

SEQ ID NO: 2 - RNA, 849 nt, Artificial Sequence
  ctg_512
uccagccugggugacagagagacuccaucucaaaaaaagaaaagaaaagaaaaaaaaagucauggagccccacuaccgaaguaggacggggacagaaucagccaugauuucgaguuaagaugcucugguuggaagccuccugcacugccuucuguaacagcaccagcuggacguugucaugaaaugucacgaguucuggguguuuccuggucugcaguccgcagcucccugccaucggccacccgaucucacgauggcacagcagacaaacucugaggagccucccaggagccuagggcuuuuccuccgcccucuucuguaagcagacuggacagcaggccccugggauguucacggggacagcacagguggcaggggggcaagcuuccacgaagcaggugaccugcccguccuggagaagagagacggagcacagccugucagcucugggauggccuucagacuggcggcagcugaccacacgccccaauggaagacauucccaccaccgugcugucaaauaaaaaccguagucacaaaggcgacguucucagaggacugugggagcaagauucaaccagaugacgagaacguccugcaccuacacuuccaccagaacgaacgaugaucacucagaauccccagagucaagucugccuuauuuuccgaguaacaacaccacuggcuuuucucuggaagcacggggccccauaacaaggugaccuuuccaaccgcugugccaugcacagggguagcagaagccuccuggaagggucuccuuccucaaacccucucuugcaucuugcagggccggggggcagacagggugacaguggccggaaaugccug

SEQ ID NO: 3 - RNA, 3448 nt, Artificial Sequence
  ctg_104447
ggacuaaaacagugacagugagugcaacucuaccuaucauguucauauucuagguagaaacuggggaaaagagaaaaggcgggaaaaaggcguuucucuacaaagaucacacauaaucuucugucugcaucucauuggucggcauuuagucacauggccauaacuaccagcaagggaagcugggaaauguaguuuuucaauggcacauugcuacccuuaauaaaauaaggauaugcuaccaacaaagaaugaauaugggaaaggccccuagcaaugucugcugcaucugccacugugguuuucucugauuuccacagacauugcucuuccacauagagcgcauucugauucccaagccaccacucccaaaucacacccaguuucugcaucuagcuugugugugggcagucugcuccaucagucccagauguggcuucucauggcuggugaccuaugaacuaaaagacaggccauuuuuuucccaaccuacccuaccuuuucuccuugagaggaaagauaaaauaaccacaauuuaaaaaugucaaucuuccauucagaaaagggaggaaagagaaacacagagaucacugguuuacagugauggacccacccugcuaggcaggagugaaaaggccucuccguccggcaguggacuggguuccuuggcuggcccaugugguccccacccacugucuccaggagcaucgccuugaacugugucuucauggcuuuuggcuccgccuucagaaggguccucccugucaaucauccuccauugccaccucugaugugggcacuagggagcugguccuucccagaggaugcaugauuuugacaccgugcagugugggauguuggaccagggaauccagcauauuuucgggcuuuuguagucucagacugggccugagauuucuuuagaaauauauuaacgguuccuucaacacuccaaugggcauauagccuauuuacuucugaucauuguaaugugcaaauaaccacagccccauuucuuuguguccagaugcaguuuuuaggcuugaauauucaucuguuuuacucacuggccucugucacugagucuguccccuuagcuauuaaauuaauggugacuaugauaacacuuuucacccgauccuugcuagugcauugacuugcguugucugcaucgaguucuuaaaagaugcuugguaaagucuugggucacaguuuuaacuucugcugggguuccugcuccacagcccucauuuagaacuguuucacuuuggcuauuguuuagcuuugggccauauuuggcuguuuuuuagcuuuguaaaaaugcucauuuaacggacacaaggccuggguuagaaaauguaguauuuccuucccccucuguuugcguaggagcuagcgcuaguggaagcccaucucucccuugcaggauuuuguggaaagugacgaaaauaacucaaaccacaucaguguucugcuugauuuccaccauucuuggaugcuauaacuucagaaagcacaugaucuaacuucccaagggacagcagggccucgaccaaaugccguugugcugagggcugccggccgccccgcuaggcugcugcacccuccccgcgcugccccacaccuugcacagagcucaggcagcauucuagcaccaucagacaaauucuucaugagucagaaaucgcaccugucucuuaauagcgaucuggccucagguggcugaaacacacaggguugaaaugcugcggugccugcgcucauugugcggugaagccauagaguccgcgcugaaacccggggcgcccgccuccuguccgcgcaggcgcugcacgcugucgccggggcagcuucacucagcuucagccucuccaucugcagaggaggaguaaucccgugcugccucauggggccguuucaagaaggaaaugagaugugguauaaaguguuagaggaguauuuaguguuauuauuaauguguuuucucaucugggaacaugauuugcauuuuaagcagaacuucaacauuuggacucaaaggagugauguggauggggagaauuucaggcaucgugcaggcuggcauuagaaaugcucaccggaaauggagcacaggcagcaacuucagccccuagaauucuuccuccuaugucacuuccgucccucaaacccuuucugcagccccuguccucucuccugcaauggcagugagcagguggucuccuggugucucuggaaaauucccauauggaguccugguugagaugagccagaaggcuaggaaggagccuacagucccggugucucagcgcccaggagguuuuucuaauggaguuggcaugcagaagcuggggcauacccugggagcagcccuguggcuggaugagggauggacagcagcacugggcggguggagccgggugcgagguccccacagccugccucccagucccacuggccagcccggggagggugcugaagacaggcuuccucucuggucaagcucuggcuagcuuggcaagugcaggcaggacccuggaccugccauauggugcacauuuugaggacaucauuagcagguuuggaagguugugauucugguugugggagagaaggaaccugggaguugagcaggugguggguggagcuaugcaugcaacugagugugaggagugcuuuggccaugcuagggucuccucugggcuggaagccuugucuucaagaggguugggguaggaggcuuuuuauuauagugggaagccucaagccuggaccugggaaaacacuuucuggguaugaggaauuaaaauaaaccuugaagcacuauuuaggaagaacauaguaucacuucauagauauuuuccccccuaaaugguuacaaaguaaaauggcagagguacaaagaggaaaaugaaaaccacccaaucccuucccugagcgauggcccuguuauccuugggcucuguggccaucugaccucucucuggggacagaaguacaugaggagaggggccacuucauaagacggagaugcucucuuccuguucccugccucugcaucgugcuggggaugucuguccucaucaacuaagagacccauguagaccgcagcucugagaucuucuauuuuuguaaacagaacaguuuguagggagaugugagauguuagguggugcuggagagcccgggcuuugucuguuuggacaaggcuccaucaaggcauccuuuguccgacggugaggcaucuugagggcucuggacccagagcugguccugcaagaguccugucugagagcccaggccccacuccacuaggagggacaggagggaacagagcagagcugagucccuucaccuacccaaacccauacauguuucuagagcagaguaacugcuugugaaacagaccaucagagcacagggcagcaccgagcugcguucugcagggcuc

SEQ ID NO: 4 - RNA, 456 nt, Artificial Sequence
  ctg_73782
guuucuuugggucaaagggagcucucauugcuacuucaguguuugcagugugcagaguuguaaaaguugagacuagccacucaaaaugcgagcuucugguccugacauuucacggggaaaggaaggcucaugaaccgacugagacaugguugagcggcuaagauuucuguagcuaugaugcugccauccaggccgacuucagacuugggaaguuuccaugugaguuuuuggaagguuuugggguuuuguuuuugauggaggaaaagaaugugaaagaguguuugcaccaaauagauuaagcuguuaguaaacaugcacccuuuaaauuaacugaugauaaggacaaagaaggugcuuugucgucgucaacuggauuugucugaggucacagagacaaacaggugaaggggcagucccucuucaacucccuaauggaaagcagagaguaaaccguga

SEQ ID NO: 5 - RNA, 424 nt, Artificial Sequence
  ctg_2815
cagaaauagaaaaauuaguccuaaaaucuguauggaaccgcaaaagaugcugaaaaaccaaagcaaucuugagcaaaaagaacaaaccuggaggcaucagacuaccugaucuuugauaaagcaaacaaaacaaaguggggaaaggauacccuauucaacauaugguccugggauaauuggcaagccacaugcagaaaaaugaaacgguucuucaaaagguuaaauauagaauuaccacgugacucaguaaauucacuccuauguauaccucaaaaagaaauuaaaacaaggugccuacacaaaaaguagcauacagguauuuauagcaacaaaaaguaggaaacaacagaaauguccaucauuaaugaaauguggccuguccauaaaauauugacaaugaaaaagaaagauauuaauacauacu

SEQ ID NO: 6 - RNA, 346 nt, Artificial Sequence
  ctg_61528
aggaggucacugucgacuucgagcaaaggagauuccugaaccaaaacugagccuagaagccaucaacacauaaguagaccuggccuuauaaaauaugaaagugcuucaugcugacaguucuguauugauggggcuguagaaaaacauuuguaagaaaagugagagauagagaguuaacaucuagaguggaaaggagcaauuaaaaucaauuaacagucaaauaguagaaaaauggacaaaggcuaugaacagaucauucguugaacaaauacaaauugccaaaaaaagauaaauaaaggacaacuuuaaaauucacuaauauucagggaaagcaacuaauguaaca

SEQ ID NO: 7 - RNA, 521 nt, Artificial Sequence
  ctg_119680
agagcucacugcaagacagaugagaaagagguuguuuuaagaugaagaaaaggaaugauacuugauucaucaucuuauacaugauucacaagaaaaauauaaugucuuuauuuuucagaggguuuuuuaaaauuaagagaugggaucuugcuacguugcccaggcuguagugcuguggcuauucacaggcauggucauagcucacuacugucuugaacuccugggcucaagccauccucuuaccucagccucccauguagcagggacuacaggugccugugaccacaucuaguuuuccaguggguuuuaaaaugaccuaaguguuugaggagaggaacaggcuggcacugagaggcaugacuaaauugugagcccuccuugaggacuggagcugugucuuauauucccagcauaaagcccagugugaagcagcacacucugcauaguggucaaagcagcugauaaauauaaggucaauaucaggacacucugacagccucacacagccaacaaacacugac

SEQ ID NO: 8 - RNA, 333 nt, Artificial Sequence
  ctg_36195
augugaaauaaggugauaauacauuccaaaguauauauuuuuucaacuuugaauauaucugguguauuuggaguaauaucugaguaaauacacuuauauguaagagaaucaaaggaacaagauauuauuuuauauccaaggaaauuaacacuuagaacauaaauacguauugcauuacuucauauuaaagaaauguuuuacaaaagaaaauaaaggagcuuauuuuauagccccauuuccacaaauaauagcaaagguacauacacauaucuaauguuuuacacacucauuauuguuucucuuaaaauuuguugcuuauacuauuuuaaaagg

SEQ ID NO: 9 - RNA, 302 nt, Artificial Sequence
  ctg_123090
gaacaaucuucuauucaaucagggaaucaggcagacacuguugguugcuuacuuggucuucauuuuuaccgccuccuacccccaugcuauucuuugccaacagaacccuucucuuguucaaggcagcauuagucuuagccaaucaacgauaaucuuuuuuccuauuuuuuuucuaucucccuuucucuuaggguuggccauguaaccccgguccuggcaaauaggaccuuuauaauagucugaaagaauaagggguggauuauugauccugggaaagcuuuugguucuccaauaaaaggggc

SEQ ID NO: 10 - RNA, 435 nt, Artificial Sequence
  ctg_17297
gauugaaucacgugcuauacuuuacuguagucccguucucuuuauauuuuacuuauaaacuaaaguuacagaguaauuaucaaaacuccauauuuguguucugaaauuggcagaauuugaauuugcaguccauaggucagcagauuucucugacuuuccuuauuuucuuugcuuagccacagaaaaacagaaaaaucagugaguuuacaugcacagaauuaaauguacagacuauucucugaaagagcucaguucuaugugaaaaugaauaauguaauaaauauucuuaugaguguaguaauaguagcuauuguuuauugaguacuucuuaugugcauggcacuuuauacugcaugcuuuaugucauuugagccuaacaaaauacuaucauuauuauuaucucauuuuuauagauauuuucuuuuuuuaauaaac

SEQ ID NO: 11 - RNA, 2889 nt, Artificial Sequence
  ctg_117356
aaagugauugaaaggccaucacugaaagagugacucacccaggugcagaugagacacagagcucaugguugccuccugugaagugcaggaaggauggagcagacagacccaaaagcacuggccucccagaugaaucaggagggaugugcaggcugggugacgucugccccggaaccaggaaagugcggcugcuuccuccacaccccgcccgccgcccaggacccagggcccuucugaaaaguggagccucccuacacccuacgcacacggcggcuguuccccacgacacagcggggggcaaaugaccaccaggagccagggagucccagggaaaggacgcagauggcucguguccuggaauuucaugggagccauuugaaaucaucagcaaaugacgcccgagucuauaagggaccucagcgcaggggcuggaugagacagggugggcagccacaaaacaggguggugaaggcccgaggccaggagggcuggguuagcacucggccaccagagggaagcaagcaagcucccucccucucucucuuucucucugucuaugugucuuucucucucccucucugucucucuccaucucucucuccacccacaccuccugaggauuguuggguuuucccauggacuaggguuagacagccagcgagucugaacggaaggcgccagguguggcaguggggaagugagguggggccuggaacacaagggcaagggccucugggaggaccgcgcaggcucggcacacugagcggacugacugcagacccuccucccacaccagggggaccaccucucugugcaaagauguccucacuugauauucucaauucuuugaggucaagguuuuauuaacacucccugaaaagugggagagcuuagugauaagacuguaucauuuuugcaagacacaaagcaagcacagcuggauggcaugugccagcucccucaccaugcugagcacuuuuacucacaucucaccccugcagacagaugccaguguccucacucacccacuggggcagacauuauuaguugccaaugcaagacccauuuccucacguccuaacuaauagaaccccaacuguguccaggcaggugacgugcccagcuaaaaauacucaccuuuucagacucccuugaaguuaguagcggccacgugauacuuccaacaaauaagacauacacagagaucugcuggggcuucugugaaaacuuuuacuucccugaaacggguaccaccuuccuucucccauucuuucuuccuccuugaagcacagaagcaaugccuggagcugcagcggccauuuugugauugugaagcaaugaccugaggaaugaaggccaacaucaaggauggcaaaacagagagaggaaaagagcaauuguccccugauggcauggcugggccacugccccaacccugugaggccaugugacuccuuauuacaugagcacaggcuagucgggguuugugcuaucugcaggugaaugcuuggagauuguacaaaaguuuauacuggaggcuuuggaauucaauagaaguagaccuggguuaaagucaggaggauauuuuggcugucauugguauuguuauugccgauaaggacccacaaggccaccacuaauggggccuccagaccagagccguguugggaguuuucccuugucccuagagccuacgcuuguggccacauuccugaucaugaaugugacaugaucauggaguucugugggguuugcugaugaguugggcaccguggaggcagaaucucggcuucucuggaccccagcuguuguuuauuuguuugauguccuuuuguuuuuacuaaacauuaggaugucuuccgggucuaaacuucucuacucccagcucaggguggcacgauggccauuagauggaguuggaaugaggaggaagagauaucccacuggaaaacuuuuucugacucugauaacaugaccuuguaucauugggguuguggggaggacuuccacauacauagaacacuauuuugaucagagaaguguuuuauuuuucuuccuguuuuacaagaaugcacacucaguauaacugguauuuguaugaacucagucacccugaacccaaauauugacauuccaggugaauuaguccugcacucgggucuguuggcucaccucaggugccagcauguacucgggaaagagugccaggcccaggugaagagggccuggggcuccugaggacggcugcccaaagcccaugacccugcucggcggggccccagauaaauuacuggcgauucuguccguggcccucccucugccagcacucagccucaggccaguccuagugcugcauuccagcugauggaacagcaggacacacggccacugcucugcuggacagccuuggcugcucucguguuccugcuguggggcaggaggcagggcagagagacugagguuugcaacucuaacgacagagaaccagauugacuguguuucgcccccagaucuagcucagcccugcuggguuuuccacuccgaggaggucagcgccaaaguguuaauuuuguaucucuggcuagaggcacagugccugcaugcaguaggcgcucagaguuuguuucauugagaauuuuuguacuauuugaaauaccgacauacuuuugucuuugaagaaaaggguccuuuauguuauuuaaaugcaaacagauaccuggugaggcuaaaaucuuucaggcacuugguuuaaaacgcuucaggcuuuuaaaccauucacaucacaugguguuugguguucaccgugggcacauuuuaggcagaguuugauacacacaacacacacaugccacacacaccacacacauauacacacacaccacauaccac

SEQ ID NO: 12 - RNA, 286 nt, Artificial Sequence
  ctg_29077
gaacaaacaugcucugaugcucuagagaaacauccauaaaaagacaaucuggaacucaaaaguggcuaauaccuuuuucaggagacaggcagaaagguaagagagccauggauaauugucuccuaacaccauucaguucaugcucucgaugcuucccuacaggauaaucucaggcuucccaaccaggcauucaaagugcaggcauuuggucugggauacaaaacccagccucaguuuaccugguuuuuuuguuugcuuguuuguuuguuuuugagacagagucuca

SEQ ID NO: 13 - RNA, 297 nt, Artificial Sequence
  ctg_57223
gggcagauuucccccuugcuguucucaugauagugagcucucaugugaucuucuuguuuaaaagugugugggaccucccccugcucucucuuccuccuucuccaggcauguaagaagugccugccuccccuucaccuuccaccaugauuccuaaggcgucuccagucaugcuuccuguacaaccugcagaacugagucaauuaaacuucuuuuuuuaaaauaaauuacccagucuuggguaguucuuuauagcaaugcaagaacggacuaauacacccaccucuacaaaagaaaaaa

SEQ ID NO: 14 - RNA, 2369 nt, Artificial Sequence
  ctg_111348
gcagaaugaggacagcggacggaugaggaccuggacuuuaccagcgcacagacagagcaaaaggagacaggagacugacaggacagaaaagagcuuuugcuuuuucuacauuuuguuuuuguuuuguuuuuuccugauagucccacaaagcugaaggucggcgggucggguggagcggguuuaucuugcugcuugggcugucucagguggcaguggcuucccuccgaggccacucucugcugccacugcucggcccaggccaggguucaggcuagagcgcggaugccgggaaguugcugggccggaccucgcggggcuccgaggagauggagggcgucccgccuccacuguuccuucuucccucuggguccuucuuuuccggacgggauggauggaccuggaggcgggagggaccugggccggggagacaagaaggagacgggcgggcaaccucacauaaguugggguugcugcauagcuucuuggugcgcugaaggguaccacgauguguagcuggggauauaggagcccgcguuuccuccugagcccuucccggaugaagaguuaggguuccagccgggcggcacggguggggagccagcagacagggcccgaccguuggccaacgcacuacccuccagagccgccccacccugcuucaucagcuucuugaacuuggaucgcuuguuuuggaaccagaucuugaccugcaagagcggacaacaacgugacucaggcugggaccacugagccuaccuaggucacagggaggaaaaaagggaccgcagaacagcagagauaggaccucccaacccugcgccagcuggcagcucccugccagagaagcagagacaucccuccuuccucccuucccccauaagagccaaaggcucgaaggccgaaccgcccgccgccgucagcccacgggggcagcucucagagagccagggaggaguugugaugaggggagaaccgggauccaggccuuagccugccaggggcucaguccgaccguggcuaccgccugcggaagauacagcgccugcauaacaccgagccagcaauuccaauaucaacaucaauaaaaagaaucgaaaaccuaggaaggaguggaggaaauuuauuugcaacgcgacccaacuuuucuaugcgccaucuuuucugcccuugaguugggacggacuaaggaggccucaagguuuugcugaacuucucuuuggauucaaaaccauucuugcacuagugucuguuugcuuucuccagcucugcuccaaaucccggaccggacgucggaaacccgcgcccgccgccgucaggaacgcucacgggcuccccggagucuuacagaaagccgggaaggaaggucccguuucuucucuaaggucugggacuugagaggugagagggcggaggaaaggaucuccccccacccccaaccgguaagaaggaggaggaggagggaggcugaggcgcggacccaggccccgagugauuagucacacaauuuaaaggcaaaggagggauuaagcccggagucuauuguuccaguuguuuaggcacgugugaccucugaguucccaaacaaaucgucuggggaaauccuuccugcgcccuaaucagauuguccagggacagagggccggacggcaggcagcucgggauaccgagagcagugacagagcuagggcagagggaaugagggaggaaggagacacgcugagagccagggguggacacaaacacauacacacagugacaccccgacccugagucccagcgagcaacaaaugaagacccgggcgcaggccggccgcggaaagccugcguggcagaacggagcggcagcggcgagguaccugagucugugugagucccaaagaggccgcgagcuccgcccucuccggcagagcuagguacugaguuugcuggaaccuccgguucaaagccugcaacugcaaacuggaauaaaucguccuggguuuacggaucuuuuuucccuugccauugaagcgcacuucaccgccuuccaccaccgugcucuucuccgaguccgccccuggagggacagcagaaguagggcccguuguuaauacgaggggccgccuuuagugcgaacagccgagagcgaguggcuaccgaaggguggcggaggcagcaccagggagagaacguucaagaaacucugaaagccagggaagggucccccaaaauccgagggccugucccgggacgcugcaggagggauagagccguucccaggagcucagcuccugccuuugcagggaaaguuugcuauuuuugcaggcgguaguugagguuuaauuacccuuaauugcauacauuguuuauagcgcuacgcaauaaauaau

SEQ ID NO: 15 - RNA, 526 nt, Artificial Sequence
  ctg_105149
auggcaaccagcaaugaccagcucaguggcuggacugagaagaagcuccaaagcacuucccaaagccaaacuugcaccaaaaaaaaggucagggucacuguuugguggucugcugcuggucugauccaccgcugcucucugaauccuggcaaaaccauuacaucugagaaguaugcucaacaaaucaaugagcuacgccaaaaacugcagcaucugcagcuggcauuggucaacauaacggguccaauucuucuccacgacaacgcucaacugcaccuugcgcaagcagcgcuucaaaaguugaacaaauugggcuacauaguuuuuccucauccgccauauucaccugacgucuugccaacuaacuaccacuucuucaaguaucucaacaacuuuuugcagggaaaacacuuccacaaccagcaggaugcagaacacgcuuuccaagaguuugucgaauccugacgcacagauuuuuaugcuacaggaauaaacuaacuuauuucucauuggcaaaaauguguug

SEQ ID NO: 16 - RNA, 200 nt, Artificial Sequence
  ctg_61472
caaaccuugugaagaugagcguugggaaaggaguaggaaggucccaauuuggaauuuugacauccuaaaagccuugggugccgcagaaugaguaagcuggaggguacuguguaaagaccucggcuacugcagcagagaaacagaucaaaaaagucaagaccggcgagaggaaagugauugaaaggccaucacugaaagag

SEQ ID NO: 17 - RNA, 1197 nt, Artificial Sequence
  ctg_23999
ucucuaauaguuguaaggauaaaauuugcaauaauucuuuuacaguguuacagaauucuaaugugaauaagaaaaaaaggaauaagacuggaauaccaagccaugaacuuggaaagaaauccaaacuggcauacuggugaauacuacauuuucuugaaccaaugugccaacuuggaacaauucuuuaacuuaccuguuucuauuuccucaacuuauaucuaucgguccucaaaauagagacaugacaaacauacacauucaauaauuauacacauauacacauauaccagaugcuguagcauuuacaaugaagucacccuuugcaaaucauuuucacauaugucugcuaguaguuaaaaagauaccuaacuuuauuggaggaaaaaauaaaucaucuauuucugcauaauuaagcuaagcucaaauauguuuucugaucccaaucuaauuguauugauuucucacuuugccuaagaccacaagaaaacauucacuacuuuugccaccuuuaaaaucuuugauccugaucuucuauguuugauggugccuacggcucucuuaugcucaugacauuuuagugaacuucacuaggugacuuuguuggaauaccucuuggaaacaacuuacuuuauagaaaauaaguaaaacauagaaauauagcaugcaagugaagagcauggacucugggaccauauugccuaauucuaaugguggcucagccucucaggagaugugcugaugggaaacgucauuuaggcucucucuguguuccucagcuuccuuaucugcaaucuggggauaguaauuguacccaacucacagaguugauaugagaauuaaaugaguuuaaaauuagaacagugccuggaacucugaaguguuggcuugcuauuauuauacuuuacauaguuaauauuauaauucagcugggcuugaccccaguacuuucuacagguguaaggaugagcaaaccccuaccccucucaggcucaguguuuugaucaguaaaauugaggugggaugaaugaucucuaucccugucauuugguuaggguuuacugacaaugccugcggcagugccaugggaguauaaauugguaagauguuauggaaggcaauguugcaguaugguuuauaauguaauggguucauacuuuuagucuugggaauuuguaauguagguaauuuaucuuacaa

SEQ ID NO: 18 - RNA, 1289 nt, Artificial Sequence
  ctg_81545_37852
aaaaaaacgugaucuaauagacaauccagauauaaagaacacaaacccuuuucccuacaauuuaaccaaaugcuaaaaugcaccaggaaacacaggauacuacagagauaaagauuuggccagcagauuaucaaccccaggaaaagaaauccauuucuguagcaugcuaugcuguuggaggcacacgucacacuucuuaauucaaggacagaaaagcacaaacuaaaaugcacacuggaguaugacucccccuucucuaucugcuaaagcgcuucgcaugugacuuucaaucucuaagcugggaaucaaucaggcagcccagccuccuauauuuucuaguuguauugcuuugagcaucaaauuuaaccucugugcaccucaauuucuucagcuggagacaaagaaaguggcugcacuguggcagaguguuugcaaauguuaaucaugguuaugguagcauucuaccuugucuuacagaugguugccuaaaaagguaggaaacuguucacgaaaaagucucaaccgguccuagagugaucagaaugacagcgcugaaauaaggagggcaguaccucuuauuccagagaaguggggagcagagaggaagauggaguggaaaggggcgaggcaaggcccuccugaaauaccucaacccaaaucuucaagaaauccccaaguccucacagugcuuuuuguggauuuuuguggaaaccgguaaaaggggcugauuugcugaccccaguggguagaaaacagagacugucaagagaacagaagagaaggcagaaaggggauggggaagugggguucgccauguucacgagcuccuggagccacagggccccccaggaacaacagagcugagacuggguggccuuguuucuggcccaauucccugggaccaccacccuggaaggacacuugagcccuacucaaggacccaccacaacaucucagcauugucgguccuuaaaugccaagguuugugcucaccauuuccauccuuaagauucucacagggaagaacccacacaacagcuuugucccugcgacucugggugaguuucugacacuggccugggcucccguuuugcccgucaccaacuucucacaguauaacuauccaagagaaguagcauuugaugucagggaaucaaaagggcuaaggcucgggaggccaaggcaggugaaucacuuguaaucaggaguucgagaccacccugaccaacauagugaaaccccaucucuacuaacaauacaaaaaauuaaccaggugugguggcaggugc

SEQ ID NO: 19 - RNA, 3923 nt, Artificial Sequence
  ctg_PCA3
acagaagaaauagcaagugccgagaagcuggcaucagaaaaacagaggggagauuuguguggcugcagccgagggagaccaggaagaucugcauggugggaaggaccugaugauacagaggaauuacaacacauauacuuaguguuucaaugaacaccaagauaaauaagugaagagcuaguccgcugugagucuccucagugacacagggcuggaucaccaucgacggcacuuucugaguacucagugcagcaaagaaagacuacagacaucucaauggcaggggugagaaauaagaaaggcugcugacuuuaccaucugaggccacacaucugcugaaauggagauaauuaacaucacuagaaacagcaagaugacaauauaaugucuaaguagugacauguuuuugcacauuuccagccccuuuaaauauccacacacacaggaagcacaaaaggaagcacagagaucccugggagaaaugcccggccgccaucuugggucaucgaugagccucgcccugugccuggucccgcuugugagggaaggacauuagaaaaugaauugauguguuccuuaaaggaugggcaggaaaacagauccuguuguggauauuuauuugaacgggauuacagauuugaaaugaagucacaaagugagcauuaccaaugagaggaaaacagacgagaaaaucuugauggcuucacaagacaugcaacaaacaaaauggaauacugugaugacaugaggcagccaagcuggggaggagauaaccacggggcagagggucaggauucuggcccugcugccuaaacugugcguucauaaccaaaucauuucauauuucuaacccucaaaacaaagcuguuguaauaucugaucucuacgguuccuucugggcccaacauucuccauauauccagccacacucauuuuuaauauuuaguucccagaucuguacugugaccuuucuacacuguagaauaacauuacucauuuuguucaaagacccuucguguugcugccuaauauguagcugacuguuuuuccuaaggaguguucuggcccaggggaucugugaacaggcugggaagcaucucaagaucuuuccaggguuauacuuacuagcacacagcaugaucauuacggagugaauuaucuaaucaacaucauccucagugucuuugcccauacugaaauucauuucccacuuuugugcccauucucaagaccucaaaaugucauuccauuaauaucacaggauuaacuuuuuuuuuuaaccuggaagaauucaauguuacaugcagcuaugggaauuuaauuacauauuuuguuuuccagugcaaagaugacuaaguccuuuaucccuccccuuuguuugauuuuuuuuccaguauaaaguuaaaaugcuuagccuuguacugaggcuguauacagcacagccucuccccaucccuccagccuuaucugucaucaccaucaaccccucccauaccaccuaaacaaaaucuaacuuguaauuccuugaacaugucaggacauacauuauuccuucugccugagaagcucuuccuugucucuuaaaucuagaaugauguaaaguuuugaauaaguugacuaucuuacuucaugcaaagaagggacacauaugagauucaucaucacaugagacagcaaauacuaaaaguguaauuugauuauaagaguuuagauaaauauaugaaaugcaagagccacagagggaauguuuauggggcacguuuguaagccugggaugugaagcaaaggcagggaaccucauaguaucuuauauaauauacuucauuucucuaucucuaucacaauauccaacaagcuuuucacagaauucaugcagugcaaauccccaaagguaaccuuuauccauuucauggugagugcgcuuuagaauuuuggcaaaucauacuggucacuuaucucaacuuugagauguguuuguccuuguaguuaauugaaagaaauagggcacucuugugagccacuuuaggguucacuccuggcaauaaagaauuuacaaagagcuacucaggaccaguuguuaagagcucugugugugugugugugugugugugaguguacaugccaaagugugccucucucucuugacccauuauuucagacuuaaaacaagcauguuuucaaauggcacuaugagcugccaaugauguaucaccaccauaucucauuauucuccaguaaaugugauaauaaugucaucuguuaacauaaaaaaaguuugacuucacaaaagcagcuggaaauggacaaccacaauaugcauaaaucuaacuccuaccaucagcuacacacugcuugacauauauuguuagaagcaccucgcauuuguggguucucuuaagcaaaauacuugcauuaggucucagcuggggcugugcaucaggcgguuugagaaauauucaauucucagcagaagccagaauuugaauucccucaucuuuuaggaaucauuuaccagguuuggagaggauucagacagcucaggugcuuucacuaaugucucugaacuucugucccucuuuguguucauggauaguccaauaaauaauguuaucuuugaacugaugcucauaggagagaauauaagaacucugagugauaucaacauuagggauucaaagaaauauuagauuuaagcucacacuggucaaaaggaaccaagauacaaagaacucugagcugucaucguccccaucucugugagccacaaccaacagcaggacccaacgcaugucugagauccuuaaaucaaggaaaccagugucaugaguugaauucuccuauuauggaugcuagcuucuggccaucucuggcucuccucuugacacauauuagcuucuagccuuugcuuccacgacuuuuaucuuuucuccaacacaucgcuuaccaauccucucucugcucuguugcuuuggacuuccccacaagaauuucaacgacucucaagucuuuucuuccauccccaccacuaaccugaaugccuagacccuuauuuuuauuaauuuccaauagaugcugccuaugggcuauauugcuuuagaugaacauuagauauuuaaagcucaagagguucaaaauccaacucauuaucuucucuuucuuucaccucccugcuccucucccuauauuacugauugcacugaacagcaugguccccaauguagccaugcaaaugagaaacccaguggcuccuugugguacaugcaugcaagacugcugaagccagaaggaugacugauuacgccucauggguggaggggaccacuccugggccuucgugauugucaggagcaagaccugagaugcucccugccuucaguguccucugcaucuccccuuucuaaugaagauccauagaauuugcuacauuugagaauuccaauuaggaacucacauguuuuaucugcccuaucaauuuuuuaaacuugcugaaaauuaaguuuuuucaaaaucuguccuuguaaauuacuuuuucuuacagugucuuggcauacuauaucaacuuugauucuuuguuacaacuuuucuuacucuuuuaucaccaaaguggcuuuuauucucuuuauuauuauuauuuucuuuuacuacuauauuacguuguuauuauuuuguucucuauaguaucaauuuauuugauuuaguuucaauuuauuuuuauugcugacuuuuaaaauaagugauucggggggugggagaacaggggagggagagcauuaggacaaauaccuaaugcaugugggacuuaaaaccuagaugauggguugauaggugcagcaaaccacuauggcacacguauaccuguguaacaaaccuacacauucugcacauguaucccagaacguaaaguaaaauuuaaaaaaaaguga

SEQ ID NO: 20 - RNA, 394 nt, Artificial Sequence
  ctg_9446
uuucuaaagcuaacugauaaaaucauuuaucuaguucagugaauaagugugguagaaguuaaaacagaaaaaaaugcuauuauuucuaaaauaauuuccccuuggacugcauuugcuaaauuagcaagugauuaaauuaauccuaguuuguauuauuugcugaaauguguguguuuccuggcauagccuaagaagaaaauguaucuacacucucauuuuguagaacucugagaggaccauccagucauugcugacaaagcccaguccuacauaucaucauaguacaugaggggcauguacaguuaaugugaaaaauaagcucaauuguccugauauguugcaguccaaaacuucauuuaugagucauuuauuuagaacucagaacacucuugcc

SEQ ID NO: 21 - RNA, 490 nt, Artificial Sequence
  ctg_63866
ugaagguauuauagucuacuaaaauacuuuaaaacaaagaaacaaugguauuuuugaguaaguuagaauagcaccaguugugugugcuuuacaaacaaaagaaacaaggaaaaaagaggaaaaaggaaaaaaaaucauuauauuugaacuagagagcaaacaugacaguuuagaauaugaagaugaaaaauaauuuggaacccaauuagaaaacuucagacagaaagagcagaaacuuuguccuuugaaacacagaugcauaagagaccgugucaaucaacucaaggaccuggcauagaagucauuuuccuugggaacuguauuuaccauauuugagaaauugaaaaauuauacucccugagaauucuaaauugcuuuauuuaucuccccaaggucagacaagugguuuucggacauggaaauaucccauucuacacuaaacaacagcuccaagcuuuauuguguguuauuacaugcagucacagacagg

SEQ ID NO: 22 - RNA, 427 nt, Artificial Sequence
  ctg_28650
uuuuauuaaccuggaaaauguguuccuuuguucugauuguguggucaccaacaacggcugauuuuuaauaugggcucucauacacccuguuaccgauuaucagacaccagauccuggucacaagaaacaacagcucccauuuguaaaugaaugccuugacauugagcuuuacccacaauggagaguugguuaacaggagguggucucucugugccugaacaauauguguguucugacaaaaucaaaucaccagcccugcaaucccacagaccucacaggcucaguuugacccaccagggaaugcugccuccauaagcuacuggccgccuuccauggcauuucuaucugugacaacugccacaagcuaccuccgcuggaugucaaacaggcacuaagcuuugugcacugggucuuugcucaaaugucc

SEQ ID NO: 23 - RNA, 245 nt, Artificial Sequence
  ctg_111158
agcagaguuacguuuuuuuuguuguuguuuuuuuuguuguuguuuuuuuagguuuccguguguuucuauugagcugcucagugcccggcuuagaagaccaggaaaaggagucacaggucguaugcuggaggcuugagccgcggcaccguggcgcggcucgccucgcugcgguuggugguggcgguggacauugcagcgcggcuggagggguaggaagaagaagauaguuuugggauagauagagg

SEQ ID NO: 24 - RNA, 1248 nt, Artificial Sequence
  ctg_25348
cggauguuccugccuauggcagcuacuucucagggcuggggaggggagcccuggugucuccaggugacuugaacaugaggucuucgccucacccaaagcccccuccucccacaggccccgugcccauggcacuccuguguccacugagcccugaaggccaccccagcccaggcuccuuccaagaggcccugguucucaugggccuuucaggugagguuggccuuaccaauucuacccagauccuccaggucagucaguggagacgccgccccucccaccaggggcugugcugggauccaggggaagccuggggcccuccuggaggcuguucugggccagcgggccugcucaccuauaggcagccccauugagcucagggugcacgacggcgggguccaugcuggcccaguggguggcggcugucagaugguccuuguugacacaguucuucaggcugucugggauccggccugggagaugcaggaggaaggaacagcuguggcucugagggggccccaggagcguggcccacuggguugaagacacgggcagcaggauucuuggcagugugcuccucccucucagccccggggcucccaaggcacggcaggguccugucucucugggcacuggcacccuugcagcguggggacccccagcccagagcacgugcccagcuugcugagggaccugucugcucagcccagcaacuucccagcagguccuguguguaagacacgacgccagggcucaacuugacuuuggccuuagaggucccugcugggggugcagugagagcaccacgggcccugagcuggcagaacaugggccuggcugccaggcggaugcgagcccaugagcaucuggggcuggcaggaagggacccccaggccugaccgagcggcccccacgcccaccugugauggcucugcggaucucccgugccgccuccucucgcaucucgauggaugccugcucgcuguaccaugcagcauggggggugcagaugagguugggugcauccuucagagggcccuggcuaaagcugggaacagcacaggcauggucagugcagccugagugcuguggcuggguacggaggggagguggcugggcacuggaacccccuguggggggcccugccugggaaccacagaccccacggccccgcucugcucggcaggcuggcugagcucaagagcacagccccgggaccaucagacacaccaggggcuccugacagcggcg

SEQ ID NO: 25 - DNA, 100 bp, Artificial Sequence
  Nanostring Probe ctg_44030
atcgccaggattctcagctgtcgagaatgtcacccgctgctgtactcccaacagcaagggaggcacctggcacgtcgtgagcaactctcgtgtatttgtt

SEQ ID NO: 26 - DNA, 100 bp, Artificial Sequence
  Nanostring Probe ctg_512
ttccaccagaacgaacgatgatcactcagaatccccagagtcaagtctgccttattttccgagtaacaacaccactggcttttctctggaagcacggggc

SEQ ID NO: 27 - DNA, 100 bp, Artificial Sequence
  Nanostring Probe ctg_104447
cacaccttgcacagagctcaggcagcattctagcaccatcagacaaattcttcatgagtcagaaatcgcacctgtctcttaatagcgatctggcctcagg

SEQ ID NO: 28 - DNA, 100 bp, Artificial Sequence
  Nanostring Probe ctg_73782
gaaaggaaggctcatgaaccgactgagacatggttgagcggctaagatttctgtagctatgatgctgccatccaggccgacttcagacttgggaagtttc

SEQ ID NO: 29 - DNA, 100 bp, Artificial Sequence
  Nanostring Probe ctg_2815
tctttgataaagcaaacaaaacaaagtggggaaaggataccctattcaacatatggtcctgggataattggcaagccacatgcagaaaaatgaaacggtt

SEQ ID NO: 30 - DNA, 100 bp, Artificial Sequence
  Nanostring Probe ctg_61528
aggaggtcactgtcgacttcgagcaaaggagattcctgaaccaaaactgagcctagaagccatcaacacataagtagacctggccttataaaatatgaaa

SEQ ID NO: 31 - DNA, 100 bp, Artificial Sequence
  Nanostring Probe ctg_119680
aaattaagagatgggatcttgctacgttgcccaggctgtagtgctgtggctattcacaggcatggtcatagctcactactgtcttgaactcctgggctca

SEQ ID NO: 32 - DNA, 100 bp, Artificial Sequence
  Nanostring Probe ctg_36195
tttggagtaatatctgagtaaatacacttatatgtaagagaatcaaaggaacaagatattattttatatccaaggaaattaacacttagaacataaatac

SEQ ID NO: 33 - DNA, 100 bp, Artificial Sequence
  Nanostring Probe ctg_123090
gggttggccatgtaaccccggtcctggcaaataggacctttataatagtctgaaagaataaggggtggattattgatcctgggaaagcttttggttctcc

SEQ ID NO: 34 - DNA, 100 bp, Artificial Sequence
  Nanostring Probe ctg_17297
tgaatttgcagtccataggtcagcagatttctctgactttccttattttctttgcttagccacagaaaaacagaaaaatcagtgagtttacatgcacaga

SEQ ID NO: 35 - DNA, 100 bp, Artificial Sequence
  Nanostring Probe ctg_117356
tagttgccaatgcaagacccatttcctcacgtcctaactaatagaaccccaactgtgtccaggcaggtgacgtgcccagctaaaaatactcaccttttca

SEQ ID NO: 36 - DNA, 100 bp, Artificial Sequence
  Nanostring Probe ctg_29077
gacaggcagaaaggtaagagagccatggataattgtctcctaacaccattcagttcatgctctcgatgcttccctacaggataatctcaggcttcccaac

SEQ ID NO: 37 - DNA, 100 bp, Artificial Sequence
  Nanostring Probe ctg_29077
gctctctcttcctccttctccaggcatgtaagaagtgcctgcctccccttcaccttccaccatgattcctaaggcgtctccagtcatgcttcctgtacaa

SEQ ID NO: 38 - DNA, 100 bp, Artificial Sequence
  Nanostring Probe ctg_111348
atacagcgcctgcataacaccgagccagcaattccaatatcaacatcaataaaaagaatcgaaaacctaggaaggagtggaggaaatttatttgcaacgc

SEQ ID NO: 39 - DNA, 100 bp, Artificial Sequence
  Nanostring Probe ctg_105149
gctgctctctgaatcctggcaaaaccattacatctgagaagtatgctcaacaaatcaatgagctacgccaaaaactgcagcatctgcagctggcattggt

SEQ ID NO: 40 - DNA, 100 bp, Artificial Sequence
  Nanostring Probe ctg_61472
caaaccttgtgaagatgagcgttgggaaaggagtaggaaggtcccaatttggaattttgacatcctaaaagccttgggtgccgcagaatgagtaagctgg

SEQ ID NO: 41 - DNA, 100 bp, Artificial Sequence
  Nanostring Probe ctg_23999
gaggtgggatgaatgatctctatccctgtcatttggttagggtttactgacaatgcctgcggcagtgccatgggagtataaattggtaagatgttatgga

SEQ ID NO: 42 - DNA, 100 bp, Artificial Sequence
  Nanostring Probe ctg_81545_37852
caccacaacatctcagcattgtcggtccttaaatgccaaggtttgtgctcaccatttccatccttaagattctcacagggaagaacccacacaacagctt

SEQ ID NO: 43 - DNA, 100 bp, Artificial Sequence
  Nanostring Probe PCA3
accagtgtcatgagttgaattctcctattatggatgctagcttctggccatctctggctctcctcttgacacatattagcttctagcctttgcttccacg

SEQ ID NO: 44 - DNA, 100 bp, Artificial Sequence
  Nanostring Probe ctg_9446
gctgaaatgtgtgtgtttcctggcatagcctaagaagaaaatgtatctacactctcattttgtagaactctgagaggaccatccagtcattgctgacaaa

SEQ ID NO: 45 - DNA, 100 bp, Artificial Sequence
  Nanostring Probe ctg_63866
tagaaaacttcagacagaaagagcagaaactttgtcctttgaaacacagatgcataagagaccgtgtcaatcaactcaaggacctggcatagaagtcatt

SEQ ID NO: 46 - DNA, 100 bp, Artificial Sequence
  Nanostring Probe ctg_28650
accagatcctggtcacaagaaacaacagctcccatttgtaaatgaatgccttgacattgagctttacccacaatggagagttggttaacaggaggtggtc

SEQ ID NO: 47 - DNA, 100 bp, Artificial Sequence
  Nanostring Probe ctg_111158
tttaggtttccgtgtgtttctattgagctgctcagtgcccggcttagaagaccaggaaaaggagtcacaggtcgtatgctggaggcttgagccgcggcac

SEQ ID NO: 48 - DNA, 100 bp, Artificial Sequence
  Nanostring Probe ctg_25348
ggccagcgggcctgctcacctataggcagccccattgagctcagggtgcacgacggcggggtccatgctggcccagtgggtggcggctgtcagatggtcc

SEQ ID NO: 49 - DNA, 100 bp, Artificial Sequence
  Nanostring Probe RPL11
acatcgatctgggtatcaaatatgacccaagcattggtatctacggcctggacttctatgtggtgctgggtaggccaggtttcagcatcgcagacaagaa

SEQ ID NO: 50 - DNA, 100 bp, Artificial Sequence
  Nanostring Probe GAPDH
cactcctccacctttgacgctggggctggcattgccctcaacgaccactttgtcaagctcatttcctggtatgacaacgaatttggctacagcaacaggg

SEQ ID NO: 51 - DNA, 100 bp, Artificial Sequence
  Nanostring Probe NOL7
gaggagctgttcatcgaacagaagaaaagaaaactccttccagacactattttggagaagttaaccacagcttcacagactaacatcaagaaatcgccag

SEQ ID NO: 52 - DNA, 100 bp, Artificial Sequence
  Nanostring Probe GPATCH3
agtctgggagcagcagtcttcgtggctggttcagggtgttttgttccgagcctgcctgcctgccggttctatacctcaggggcatttttacaaaaagccc

SEQ ID NO: 53 - DNA, 100 bp, Artificial Sequence
  Nanostring Probe ZNF2
cacgatgcttcagacaaaaaatcagaaggatcattgagggaatgccttggaaggcaaagtcctctgtgtcctaaatttgaagttcatacacccaatggca

SEQ ID NO: 54 - DNA, 100 bp, Artificial Sequence
  Nanostring Probe ZNF346
tgctggagggccaggagccggacggggtgcgctttgaccgcgagagggcgcgccgcctgtgggaagccgtgtccggtgcccagccggtgggtagagagga